(19) United States
(12) Patent Application Publication
(10) Pub. No.: US 2020/0016205 A1
(43) Pub. Date: Jan. 16, 2020

(54) Suppression of Microglial Activation with Innate Lymphoid Cells

(71) Applicant: Janssen Pharmaceutica NV, Beerse (BE)

(72) Inventors: German Rodrigo Aleman Muench, San Diego, CA (US); Anindya Bhattacharya, San Diego, CA (US); Noel Christopher Derecki, San Diego, CA (US); Pejman Soroosh, San Diego, CA (US)

(21) Appl. No.: 16/451,426

(22) Filed: Jun. 25, 2019

Related U.S. Application Data
(60) Provisional application No. 62/698,545, filed on Jul. 16, 2018.

(51) Int. Cl. A61K 35/17; A61K 38/20; A61P 25/00; A61P 29/00; A61P 37/06

(52) U.S. Cl. CPC A61K 35/17; A61K 38/20; A61K 2035/122; A61P 29/00; A61P 37/06; A61P 25/00

(57) ABSTRACT

Compositions and methods of using ILC2 to reduce microglial activation or to reduce blood-brain barrier (BBB) permeability are described. Also described are methods and compositions that use an agent that increases the number of activated ILC2 to reduce microglial activation or BBB permeability.

### CROSS-REFERENCE TO RELATED APPLICATIONS

This application claims priority to U.S. Provisional Patent Application No. 62/698,545, filed on Jul. 16, 2018, the disclosure of which is incorporated herein by reference in its entirety.

### FIELD OF THE INVENTION

The invention relates generally to methods and compositions to suppress microglial activation. In particular, the present invention relates to compositions and methods of suppressing microglial activation or blood-brain barrier disruption by administering type II innate lymphoid cells or agents that increase the number or activity of type II innate lymphoid cells.

### BACKGROUND OF THE INVENTION

Innate lymphoid cells (ILCs), as their name suggests, display features of both innate and adaptive immunity. While ILCs arise from a common lymphoid precursor and differentiate into subsets analogous to T helper cells, ILCs do not undergo the somatic recombination that underlies receptor diversity emblematic of adaptive immunity. Thus, unlike conventional T cells, ILC2s are not antigen specific and they also lack specific lineage markers that identify other lymphocytes, including T, B, natural killer, and natural killer T cells. ILCs can be divided into three types (ILC1, ILC2, ILC3) based on the cytokines that they produce and the transcription factors that regulate their development and function (Spits et al., 2013, Nat Rev Immunol. 13(2):145-9).

ILC2 s predominantly produce type 2 cytokines such as interleukin 4 (IL-4), IL-5, IL-9, and IL-13. As innate cells, they respond primarily to alarmins—endogenous Damage Associated Molecular Patterns (DAMPs) such as IL-25 and IL-33 (Neill et al., 2010, Nature 464(7293): 1367-1370). A subset of activated ILC2s can also produce IL-10, an anti-inflammatory cytokine (Seehus et al., 2017, Nat Commun. Dec 1;8(1):1900). This evidence suggests ILC2s have diverse roles in immune response. Mouse models of asthma have shown that ILC2s contribute to eosinophilic inflammation and hyperresponsiveness, and human studies have revealed increases in ILC2 counts in patients with asthma, allergic rhinitis, eosinophilic esophagitis, and atopic dermatitis (Doherty et al., J Allergy Clin Immunol. 2017; 139(5): 1465-1467).

ILC2s are tissue-resident and first shown to be enriched in mucosal, barrier, and adipose tissues. Only recently, however, were ILC2s unexpectedly detected within the context of peripheral and central nervous system (CNS, PNS) (Gadani et al., 2017, J Exp Med. 214(2):285-296). In CNS, ILCs have been examined only in pathology, with ILC2s shown to respond after spinal cord injury, and ILC3s revealed to be normal residents of the meninges and exhibit disease-induced accumulation and activation in experimental autoimmune encephalomyelitis (EAE) (Hatfield and Brown, 2015, Cell Immunol. 297(2):69-79).

Inflammation in the CNS is believed to play an important role in the pathway leading to neuronal cell death in a number of neurodegenerative diseases including, e.g., Parkinson's disease, Alzheimer's disease, prion diseases, amyotrophic lateral sclerosis, multiple sclerosis, Huntington's disease and HIV-dementia. The inflammatory response is mediated by activated microglia, the resident immune cells of the CNS, which normally respond to neuronal damage and remove damaged cells by phagocytosis.

Neuroinflammatory responses can be beneficial or harmful to motor neuron survival. Activated microglia can release neurotoxic molecules such as proinflammatory cytokines (e.g., tumor necrosis factor alpha, TNFα), nitric oxide, and superoxide (Chao et al., 1992, J Immunol. 149(8):2736-41). These neurotoxic molecules can damage or kill neurons, which can precede or exacerbate certain neurological diseases. In many studies, activated microglia have been found to be a hallmark of brain pathology.

Many diseases and physiological stressors that affect the CNS also alter the functional integrity of the blood-brain barrier (BBB). They affect the barrier abilities to selectively restrict passage of substances from the blood to the brain. The BBB disruption can lead to diseases such as depression, anxiety, brain fog, and autoimmune brain problems.

Suppressing microglial activation provides a promising therapeutic avenue for reducing the inflammation associated with neurodegenerative disease, preventing or treating a disease related to the BBB disruption. Certain therapies have been proposed that utilize compounds to modulate microglial activation (US 2011021413, WO 9945950, US 20120237482); however, compounds are known to have off-target effects. Additionally, stem cell therapy has been suggested as having the potential to alter microglial activation (Giunti et al., 2012, Stem Cells. 30(9):2044-53, WO 2011106476). The long term effects of these therapies is unclear as the survival of mesenchymal stem cells is only a few months in vivo (Volkman and Offen, 2017, Stem Cells, 35(8):1867-1880).

Thus, there remains a need for effective therapeutics that can treat diseases, such as neurodegenerative diseases, associated with activated microglia.

### BRIEF SUMMARY OF THE INVENTION

The invention satisfies this need by providing methods for suppressing microglial activation in a subject in need thereof. These methods include increasing the number and/or activity of activated type II innate lymphoid cells (ILC2s) in a subject.

The inventors unexpectedly found that increasing the number of meningeal ILC2s suppresses microglial activation. The inventors also unexpectedly found that meningeal ILC2s can be activated to produce IL-10, which mediates suppression of microglial activation. In particular, the inventors found that ILC-deficient mice exhibit disinhibited microglial inflammatory responses and increased blood-brain barrier (BBB) permeability. Subsequent transcriptomic analysis unexpectedly revealed meningeal ILC2s as provisioners of IL-10. Accordingly, rectification of neuroinflammatory phenotypes followed meningeal engraftment of adoptively-transferred ILC2s with benefit abolished by IL-10-neutralizing antibodies or Il10^{-/-} ILC2s. In addition, ILC2s from several murine tissues displayed IL-10 competency, as did ILC2s from human blood, suggesting a previously-unappreciated immunoregulatory role for canonical ILC2s adding to recently-described tissue-restricted transcriptionally-unique ILCREG subsets. Collectively, these findings show roles for meningeal ILC2s in suppression of neuroinflammation and BBB stability, indicating that ILC2-based cell therapies can be used in treatment of neuroinflammatory pathologies.

In one general aspect, the invention relates to methods of suppressing microglial activation or reducing BBB permeability or increasing BBB stability in a subject in need thereof

In one embodiment, the present application provides a method of suppressing microglial activation in a subject in need thereof, comprising administering to the subject a therapeutically effective amount of activated ILC2s.

In one embodiment, the present application provides a method of reducing BBB permeability or increasing BBB stability in a subject in need thereof, comprising administering to the subject a therapeutically effective amount of activated ILC2s.

In embodiments of the application, the activated ILC2 is activated by contacting the ILC2 with at least one cytokine selected from the group consisting of IL-33, IL-25, IL-2 and IL-7, or a combination thereof.

In other embodiments of the application, the cells are genetically modified, preferably the cells are genetically modified for increased IL-10 production compared to otherwise identical unmodified cells.

In embodiments of die application, a therapeutically effective amount of activated ILC2s is administered to a subject in need thereof by intravenous or intrathecal administration.

In another embodiment, the application provides a method of reducing microglial activation, reducing BBB permeability or increasing BBB stability in a subject in need thereof, comprising administering to the subject a. therapeutically effective amount of an agent capable of increasing the number of activated ILC2s in the subject, preferably the activated ILC2s are meningeal ILC2s.

In yet another embodiment, the application provides a method of reducing microglial activation, reducing BBB permeability or increasing BBB stability in a subject in need thereof, comprising administering to the subject an effective amount of an agent capable of increasing production of IL-10 by an activated type II innate lymphoid cell (ILC2) in the subject.

In yet another embodiment, the application provides a method of reducing microglial activation, reducing BBB permeability or increasing BBB stability in a subject in need thereof, comprising administering to the subject an effective amount of an agent capable of increasing production of Timp 1 by an activated type II innate lymphoid cell (ILC2) in the subject.

In certain embodiments of the application, the subject is in need of a treatment of a disease related to microglial activation or BBB disruption, such as neurodegenerative disease, inflammatory disorder, meningitis, stroke, neuropsychologic disorder, chronic pain, traumatic brain injury, spinal cord injury, optic nerve inflammation, a viral or bacterial infection.

In preferred embodiments of the application, the subject is in need of a treatment of a neurodegenerative disease selected from the group consisting of Alzheimer's disease, Parkinson's disease, dementia, multiple sclerosis, a prion disease, amyotrophic lateral sclerosis, Huntington's disease, and aging.

In other preferred embodiments of the application, the subject is in need of a treatment of a neuropsychologic disorder selected from the group consisting of depression, anxiety, bipolar depression, and schizophrenia.

In another general aspect, the application provides pharmaceutical compositions for reducing microglial activation in a subject in need thereof and methods of preparing the same,

In one embodiment, the application provides a pharmaceutical composition for reducing microglial activation in a subject in need thereof, comprising a therapeutically effective amount of isolated activated ILC2s and a pharmaceutically acceptable carrier. The application also provides a method of preparing the pharmaceutical composition, comprising combining the therapeutically effective amount of isolated activated ILC2s with the pharmaceutically acceptable carrier.

In another embodiment, the application provides a pharmaceutical composition for reducing microglial activation in a subject in need thereof, comprising a therapeutically effective amount of an agent capable of increasing the number of activated type II innate lymphoid cells (ILC2s) in the subject and a pharmaceutically acceptable carrier. The application also provides a method of preparing the pharmaceutical composition, comprising combining the therapeutically effective amount of the agent with the pharmaceutically acceptable carrier.

In yet another embodiment, the application provides a pharmaceutical composition for reducing microglial activation in a subject in need thereof, comprising a therapeutically effective amount of an agent capable of increasing production of IL-10 or Timp1 by an activated type II innate lymphoid cell (ILC2s) in the subject and a pharmaceutically acceptable carrier. The application also provides a method of preparing the pharmaceutical composition, comprising combining the therapeutically effective amount of the agent with the pharmaceutically acceptable carrier.

These and other aspects and advantages of the present invention will become apparent from the subsequent detailed description and the appended claims. It is to be understood that one, some, or all of the properties of the various embodiments described herein can be combined to form other embodiments of the present invention.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

### IN THE DRAWINGS

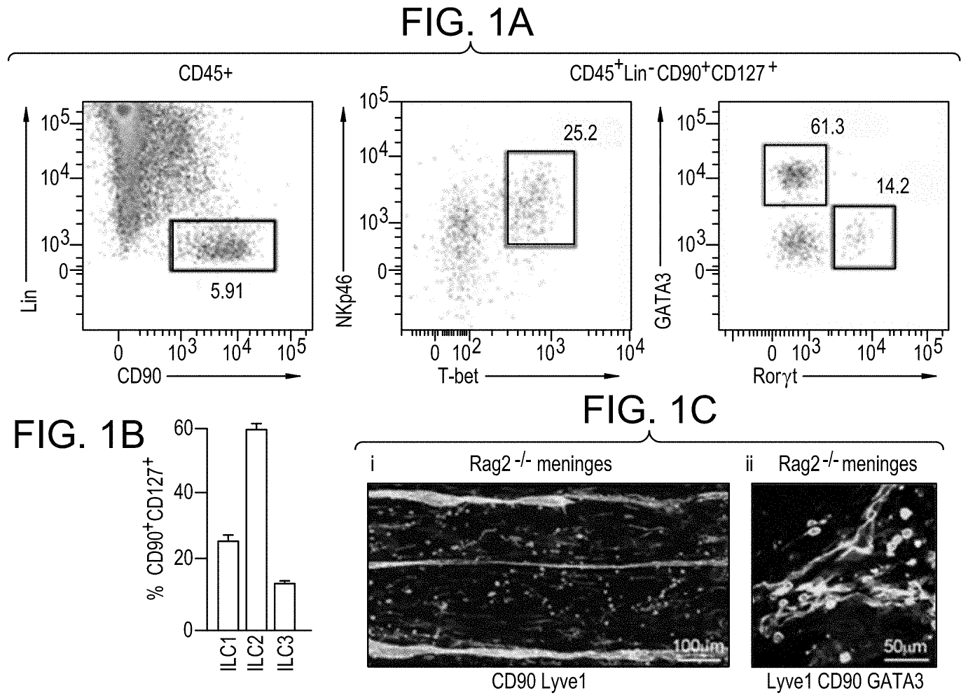

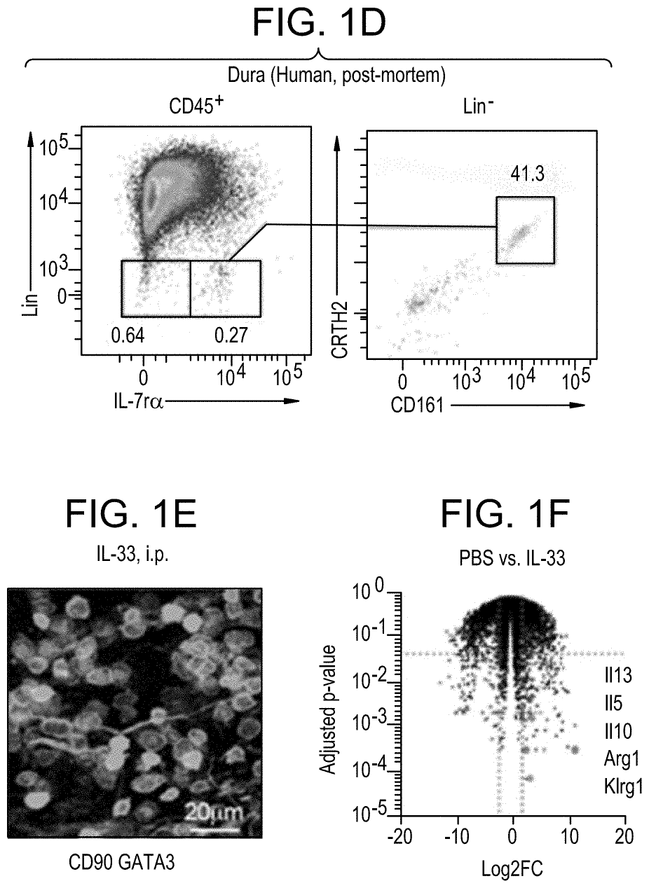

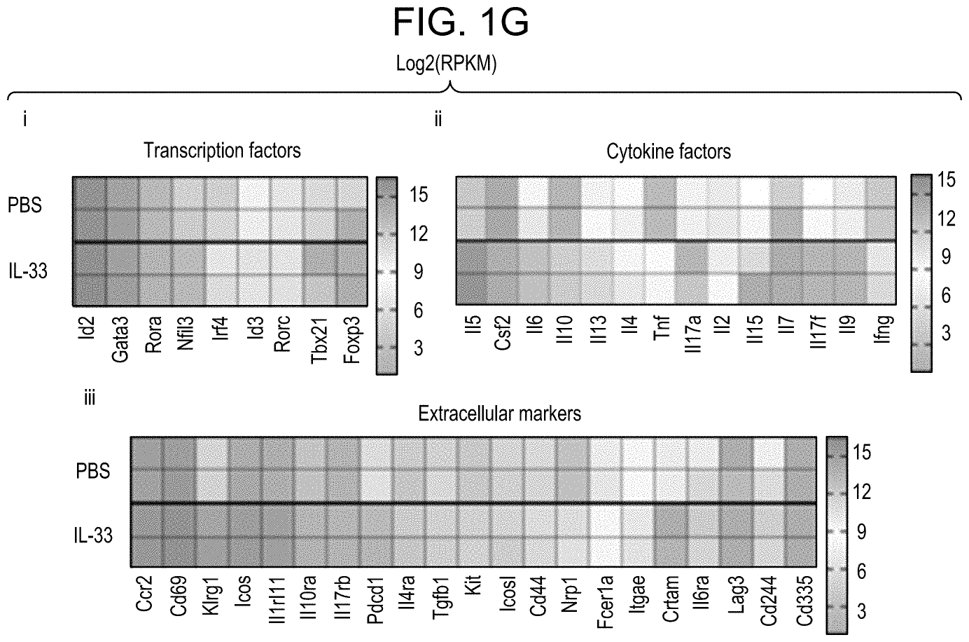

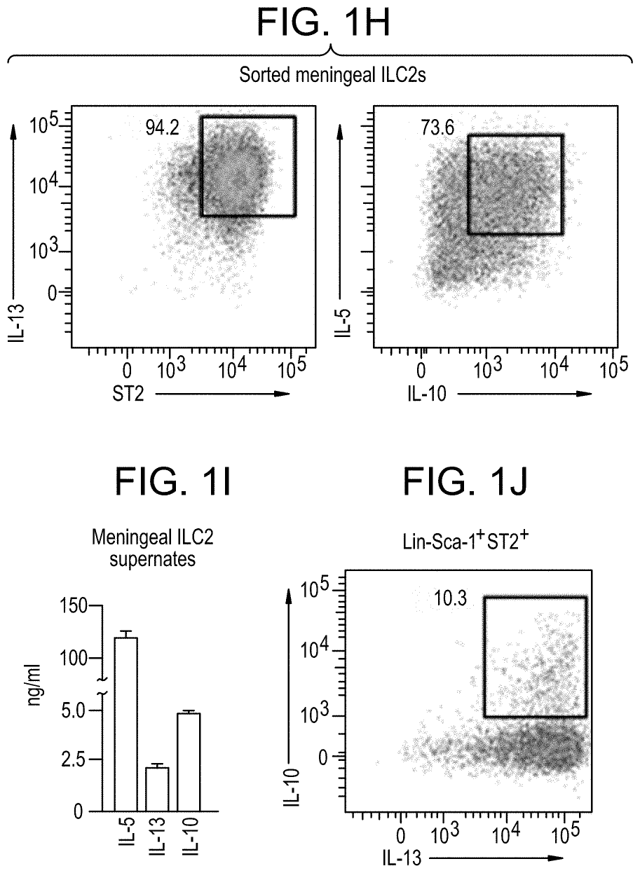

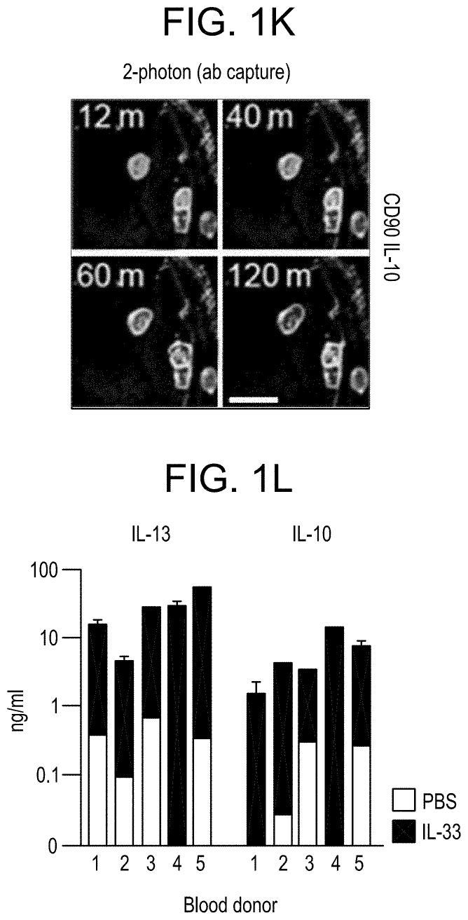

FIGS. 1A-1L show meninges are enriched for ILC2s that exhibit an IL-10-dominant activation profile:

FIG. 1A: FACS plots of Rag2^{-/-} meninges showing (L to R) gating of ILCs: CD45⁺Lin⁻ CD90⁺ and CD45⁺Lin⁻CD90⁺CD127⁺ cells expressing Tbet (ILC1s), GATA3 (ILC2s) or Rorγt (ILC3s);

FIG. 1B: Relative frequency of ILC1s, 2s, 3s found in meninges (n=4 mice×2 experiments);

FIG. 1C(i): Representative confocal image of Rag2^{-/-} meninges in whole mount. Sagittal sinus is shown, labeled with antibodies to CD90 (green) and Lyvel (red) showing CD90⁺ cells (ILCs) distributed in sinus;

FIG. 1C(ii): Representative confocal image of menILC2s in Rag2^{-/-} sagittal sinus labeled with antibodies to CD90 (green) Lyvel (blue) and GATA3 (red);

FIG. 1D: Representative FACS plots showing cells isolated from post-mortem human meninges, CD45⁺/viable/single events shown, left plot shows Lin⁻ (Lineage cocktail, CD11c, Fcϵrlα) IL7rα⁺ cells; right plot shows labeling for ILC2 markers CRTH2 and CD161, representative of 6 samples between 2 exp.;

FIG. 1E: Representative confocal image showing Rag2^{-/-} sagittal sinus following 3 d systemic IL-33 treatment; CD90 (green) and GATA3 (red) label ILC2s; 1 of 3 experiments shown;

FIG. 1F: Volcano plot comparing transcripts expressed by ILC2s sorted from PBS and IL-33 treated mice;

FIG. 1G: Heatmaps comparing meningeal ILC2s sorted from PBS and IL-33 treated mice for (i) transcription factors (ii) cytokine factors and (iii) extracellular markers. N=20/20 mice×2 exp.;

FIG. 1H: Representative FACS intracellular cytokine labeling of IL13, IL-5 and IL-10 in ILC2s sorted from Rag2^{1-/-} meninges, cultured 5 d with IL-2/IL-7 and re-stimulated with IL-33; n=20 mice, 1 of 2 exp. shown;

FIG. 1I: Luminex measurements of IL-5, IL-13 and IL-10 from supernates of IL-33 stimulated mILC2s; n=20 meninges, 4 wells;

FIG. 1J: Representative FACS plots of IL-10 and IL-13 intracellular cytokine labeling of meningeal CD90⁺ST2⁺cells acutely isolated from mice following 3 d systemic treatment with IL-33; n=3 mice, 1 of 2 experiments shown;

FIG. 1K: Still frames from 2-photon time-lapse IL-10 antibody capture movie; fluorescent antibodies to CD90 (green) and IL-10 (red) show increase in IL-10 fluorescence at 12, 40, 60 and 120 m. Representative of 3 exp. shown; and

FIG. 1L: Luminex measurements of IL-10 from supernates of unstimulated or IL-33 stimulated ILC2s isolated from blood of healthy human donors. N=5 unique donors of 9 total with data from 1 of 2 exp. shown;

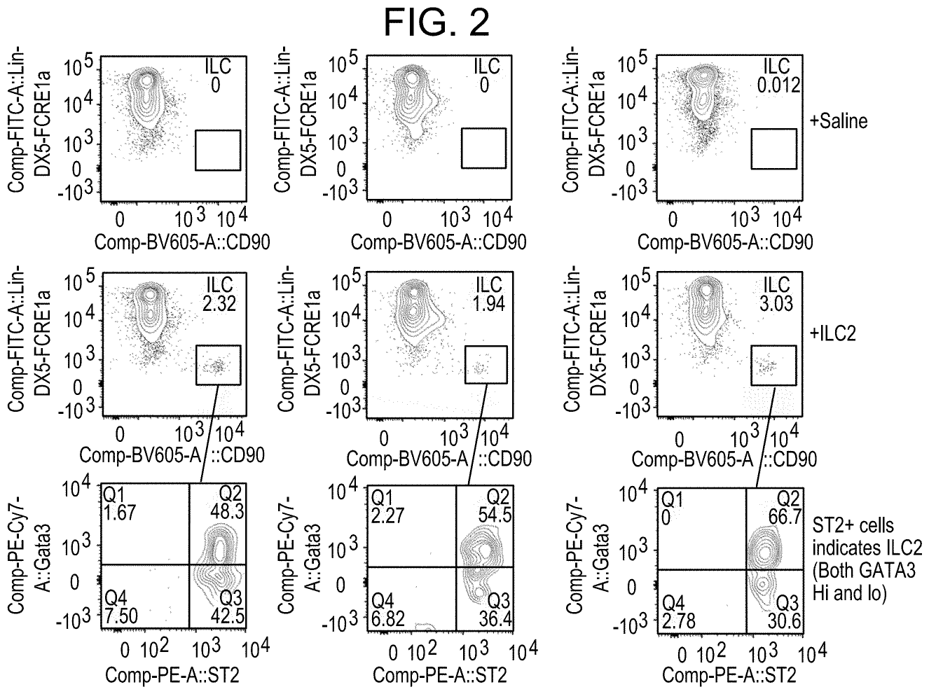

FIG. 2 shows quantification of meningeal ILC2s from mice after passive transfer as measured by FACS;

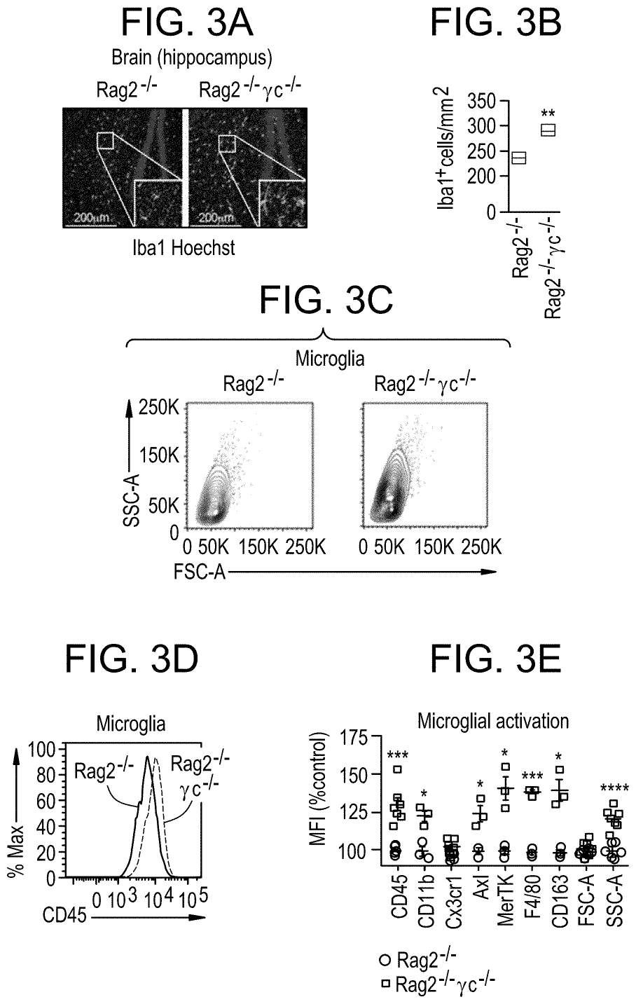

FIGS. 3A-E show ILC-deficient Rag2^{-/-} γc^{-/-} mice exhibit microglial abnormalities at baseline:

FIG. 3A: Representative confocal images of Rag2^{-/-} and Rag2^{-/-}γc^{-/-} brain (hippocampus). Iba1 (green) labels microglia;

FIG. 3B: Counts of Iba1⁺ cells/mm² in naïve Rag2^{-/-} and Rag2^{-/-}γc^{-/-} brain hippocampus; **, p<0.01, Student's T-test, n=3/3 mice:

FIG. 3C: Representative FACS plots display side scatter (relative granularity) of microglia acutely isolated from naïve Rag2^{-/-} and Rag2^{-/-}γc^{-/-} mice; n=3/3 (x 2 exp.);

FIG. 3D: Representative histogram showing relative CD45 expression on microglia from Rag2^{-/-} and Rag2^{-/-}γc^{-/-} mice; and

FIG. 3E: Comparative expression of a panel of markers associated with microglial activation in brains isolated from Rag2^{-/-} and Rag2^{-/-}γc^{-/-} mice; mean fluorescence intensity (MFI) as a percentage of Rag2^{-/-} control is shown. *, p<0.05; **, p<0.01; ***, p<0.001 ****, p<0.0001; multiple T-tests with Holm-Sidak method; n=3/3 (x 2 exp.);

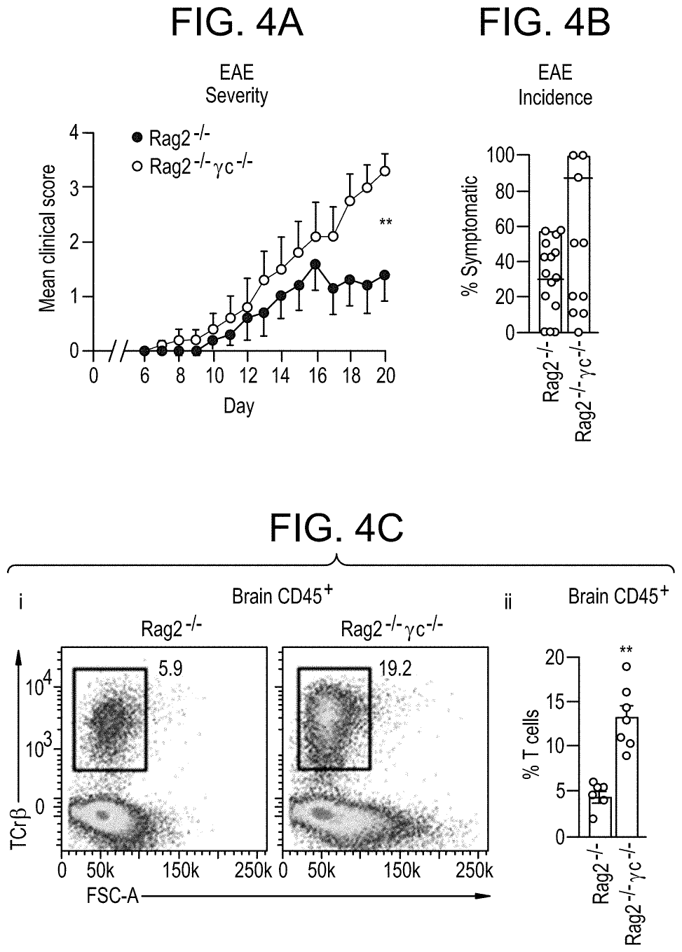

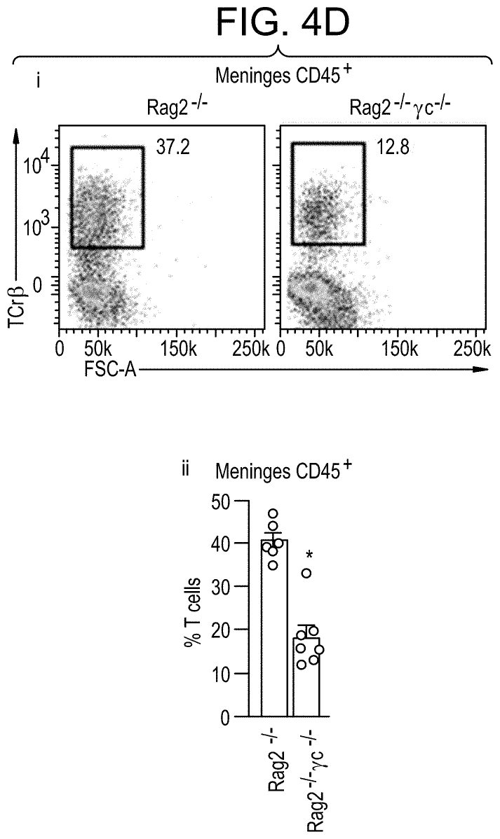

FIGS. 4A to 4D(ii) show that increased experimental autoimmune encephalomyelitis (EAE) severity in ILC-deficient mice parallels failure of T cells to arrest in meninges:

FIG. 4A: Mean clinical EAE scores for Rag2^{-/-} and Rag2^{-/-}γc^{-/-} mice; n=10/10 (2 exp.);

FIG. 4B: EAE incidence for Rag2^{-/-} and Rag2^{-/-}γc^{-/-} mice following passive EAE induction; **, p<0.01; ANOVA; n=10/10 (2 exp.);

FIG. 4C(i): Representative FACS plots of Infiltrating T cells as percentage of total CD45⁺cells isolated from brains of Rag2^{-/-} and Rag2^{-/-}γc^{-/-} mice;

FIG. 4C(ii): graph of full data from FIG. 4C(i), n=7/7 (2 exp.);

FIG. 4D (i): Representative FACS plots of T cells as percentage of total CD45⁺cells isolated from meninges of Rag2^{-/-} and Rag2^{-/-}γc^{-/-} mice; and

FIG. 4D(ii): Graph of full data from FIG. 4D(i), n=7/7 (2 exp.);

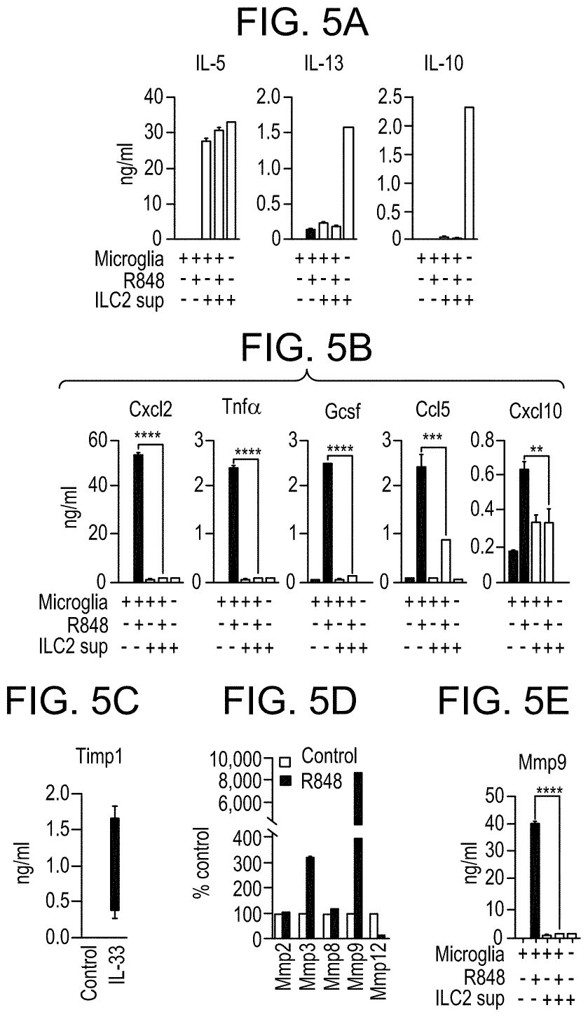

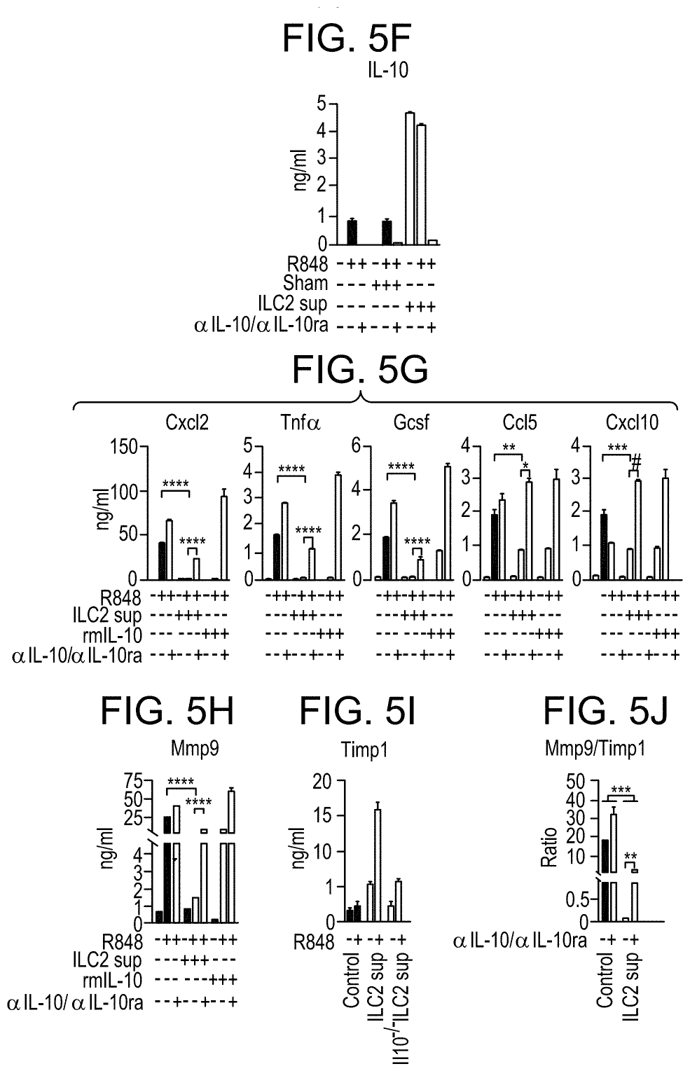

FIGS. 5A to 5J show ILC2 secreted factors suppress microglial inflammation and IL-10 neutralization abolishes suppression:

FIG. 5A and FIG. 5B: Microglia were cultured with or without ILC2 supernates and/or 1 ug/ml R848 (see matrix beneath each graph) with final supernates analyzed by Luminex for secreted factors. *, p<0.05; **, p<0.01; ***, p<0.001; ****, p<0.0001; ANOVA. 2 exp. x duplicate samples;

FIG. 5C: Microglia were cultured w/wo rIL-33 with supernates analyzed by Luminex for Timp1;

FIG. 5D: Microglia were cultured w/wo 1 ug/ml R848 with supernates analyzed by Luminex for Mmp2, 3, 8, 9 and 12;

FIG. 5E: Microglia were cultured w/wo ILC2 supernates and/or 1 ug/ml R848 (see matrix beneath each graph) with final supernates analyzed by Luminex for secreted factors; p<0.0001; ANOVA. Representative experiments of 2 shown (2 exp.);

FIG. 5F and FIG. 5G: Microglia were cultured as previously but also w/wo antibodies to IL-10 and IL-10rα with final supernates analyzed by Luminex for secreted factors. *, p<0.05; **, p<0.01; ***, p<0.001; ****, p<0.0001; ANOVA. Duplicate wells (2 exp.);

FIG. 5H: Microglia were cultured as previously but also with rmIL-10 with final supernates analyzed by Luminex for secreted factors. ****, p<0.0001; ANOVA. Duplicate wells (2 exp.);

FIG. 5I: Microglia were cultured w/wo R848 and treated with plain medium, medium containing IL-2, IL-7, and IL-33 but no ILC2s, supernates from wild type ILC2s or supernates from Il10^{-/-} ILC2s; final supernates were analyzed by Luminex for Timp1. *, p<0.05; **, p<0.01; ***, p<0.001; ****, p<0.0001; ANOVA; and

FIG. 5J: Microglia were treated with ILC2 supernates or medium, then stimulated with R848; final supernates were analyzed by Luminex for Mmp9 and Timp1. *, p<0.05; **, p<0.01; ***, p<0.001; ****, p<0.0001; ANOVA;

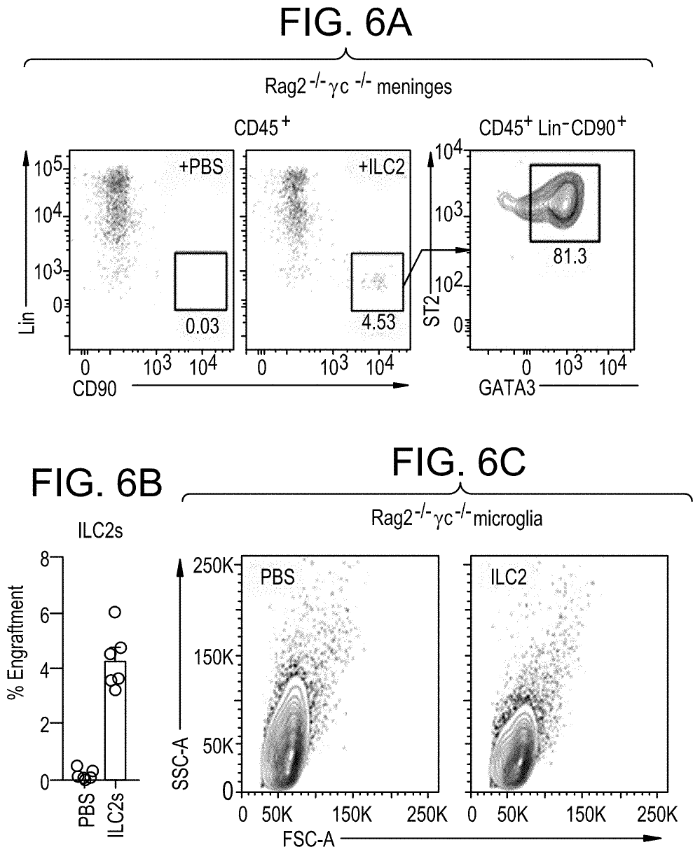

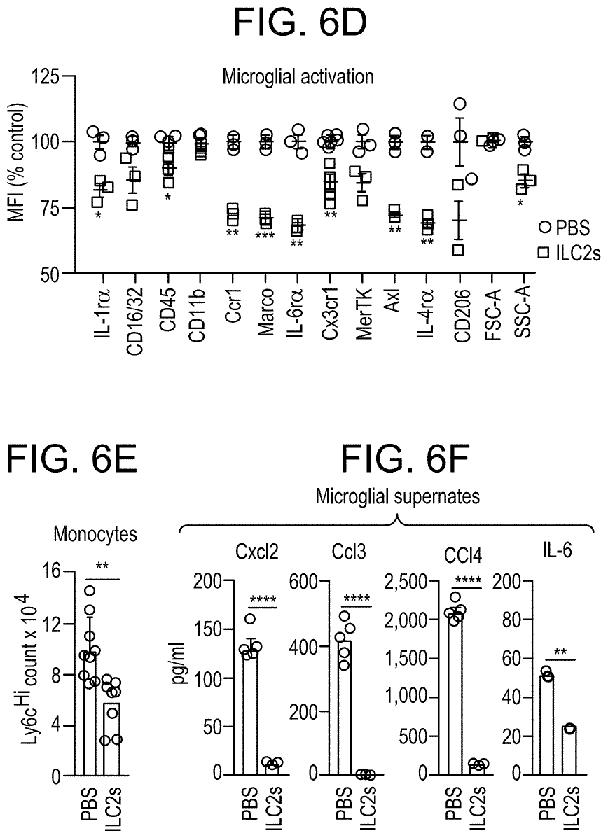

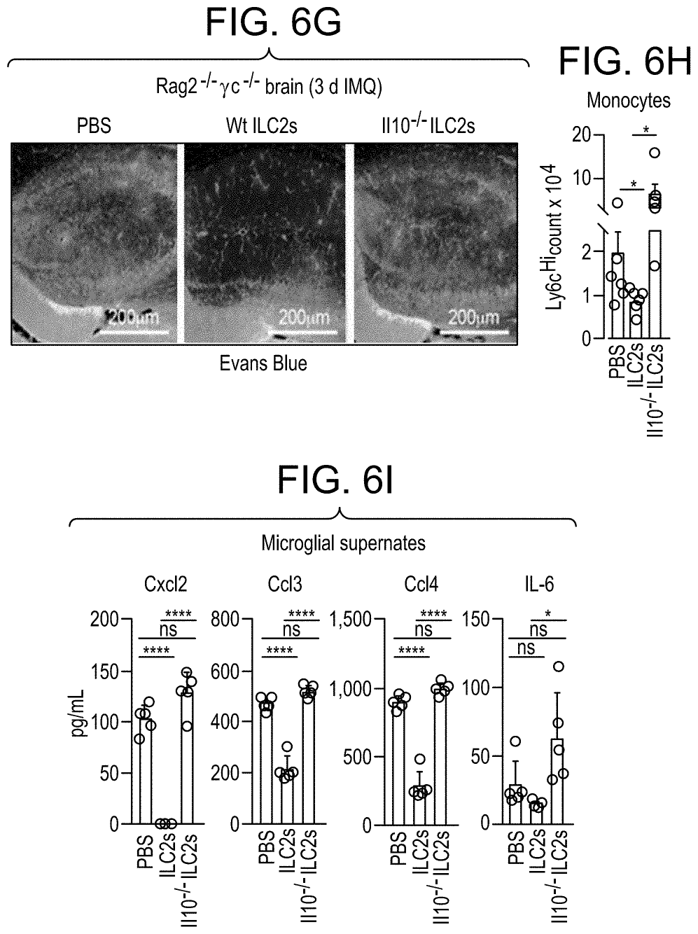

FIGS. 6A to 6I show passively-transferred ILC2s engraft in meninges suppressed neuroinflammation in an IL-10 dependent manner:

FIG. 6A: Representative FACS plots show Lin⁻CD90⁺ cells in meninges isolated from Rag2^{-/-} mice following i.v. passive transfer of ILC2s and GATA3 and ST2 labeling of those cells;

FIG. 6B: cells ILC2 percentage of CD45 from meninges of engrafted mice n=3/3 mice (2 exp.);

FIG. 6C: Representative FACS histograms show comparative expression of side scatter in microglia acutely isolated from Rag2^{-/-}γc^{-/-} mice following i.v. transfer of ILC2s or PBS control; n=3/3 (2 exp.);

FIG. 6D: Comparative expression measured by mean fluorescence intensity (MFI) of a panel of microglial activation markers in FACS of brains isolated from Rag2^{-/-}γc^{-/-} mice following i.v. transfer of ILC2s or PBS control. *, p<0.05; **, p<0.01; ***, p<0.001; multiple T-tests, Holm-Sidak method; n=3/3 (2 exp.);

FIG. 6E: Counts of monocytes sorted from brains of Rag2^{-/-}γc^{-/-} mice following passive transfer of ILC2 or PBS and 3 d IMQ. Student's T test; **, p<0.01; n=5/5, 3/3 (2 exp.);

FIG. 6F: Luminex measurement of factors secreted by microglia sorted from brains of mice in E; Student's T-test; **, p<0.01; ****, p<0.0001; n=5/5 (one of 2 exp.);

FIG. 6G: Representative confocal images of Evans Blue infiltration of hippocampal brain parenchyma in slices from IMQ-treated Rag2^{-/-}γc^{-/-} mice following passive transfer of either wild type or Il10^{-/-} ILC2s or PBS; n=4/4/4 (2 exp.);

FIG. 6H: Counts of monocytes sorted from brains of Rag2^{-/-}γc^{-/-} mice following passive transfer of ILC2 or PBS and 3 d IMQ. Student's T-test; **, p<0.01; n=5/5, 3/3 (2 exp.); and

FIG. 6I: Luminex analysis of microglia secreted factors following 2 d culture. **, p<0.01; ****, p<0.0001; ANOVA; n=6/5/5 (from 2 exp.) with similar results;

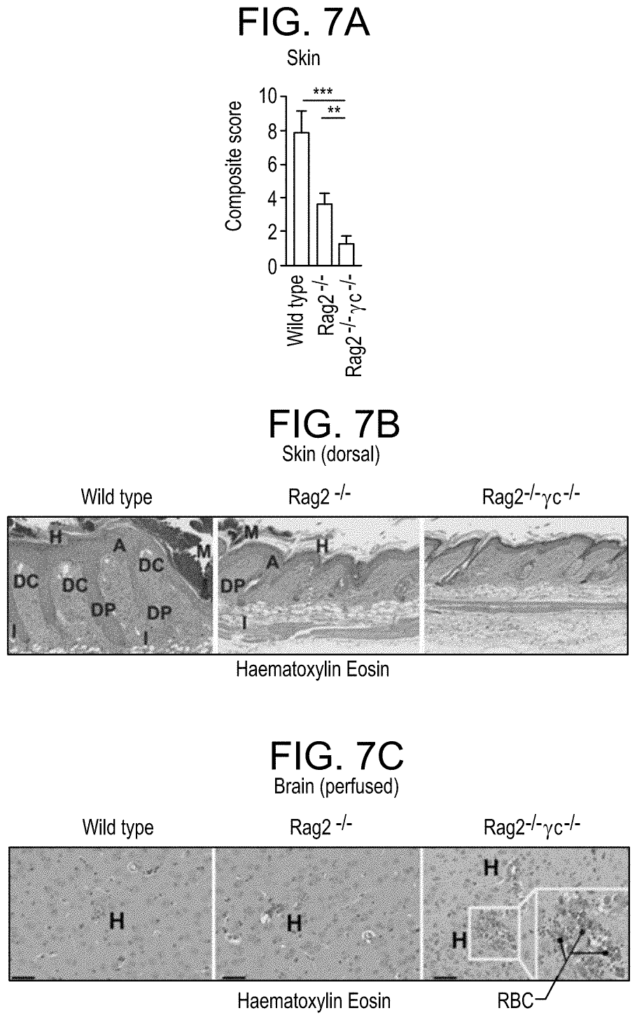

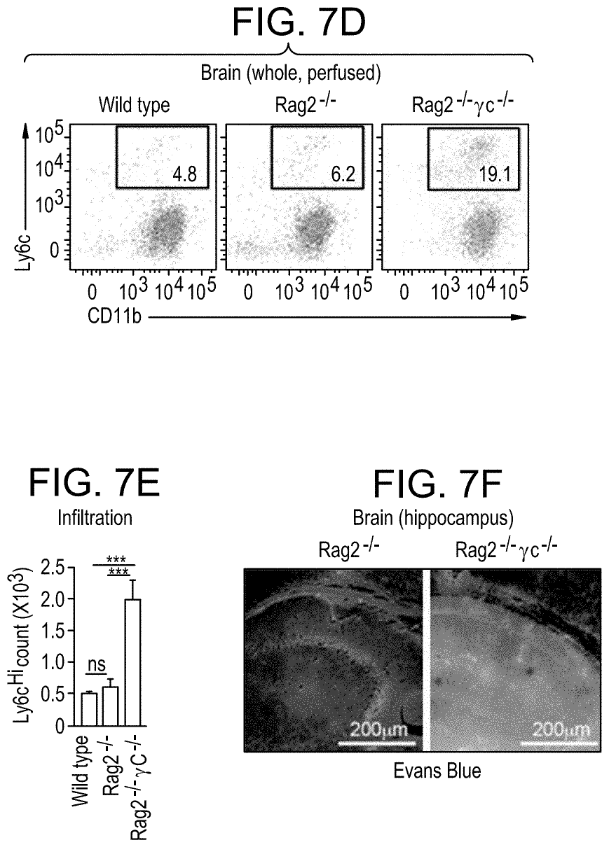

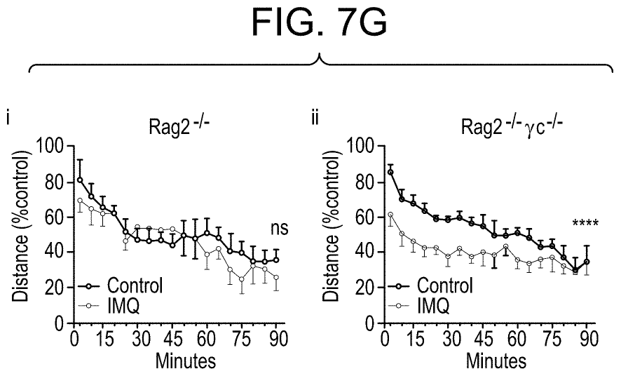

FIGS. 7A to 7G show ILC-deficient mice display decoupled' skin vs. brain responses to IMQ challenge:

FIG. 7A: Overall clinical skin pathology scores from dorsal haired skin of wild type, Rag2^{-/-} and Rag2^{-/-}γc^{-/-} mice following IMQ treatment; **, p<0.01; ***, p<0.001; ANOVA, n=4/4/4;

FIG. 7B: Representative H&E labeling of dorsal skin from three groups: marked hyperkeratosis; ‘M,’ marked Munro microabscesses; ‘A,’ marked acanthosis; ‘I,’ marked dermal infiltrates; ‘DP,’ marked dermal papillae; ‘DC,’ dilated capillaries were noted in the dorsal haired skin from Wild type. ‘H,’ Hyperkeratosis; ‘M,’ Munro microabscesses; ‘A,’ acanthosis; ‘I,’ dermal infiltrates were noted in Rag2^{-/-}. No remarkable histopathology changes were noted in Rag2^{-/-}γc^{-/-} , n=4/4/4;

FIG. 7C: Representative H&E labeling of brain sections from three groups; slight microhemorrhages ‘H’ were noted in wild type and Rag2^{-/-} and were marked in Rag2^{-/-}γc^{-/-} mice, n=4/4/4;

FIG. 7D: Representative FACS plots showing peripheral monocytes (Ly6c^{Hi}CD11b^{Hi}) content of total CD45⁺cells isolated from wild type, Rag2^{-/-} and Rag2^{-/-}γc^{-/-} brains following 3 d topical treatment with 0.8 mg/day TLR7 agonist IMQ;

FIG. 7E: Quantification of (D); p<0.01; ***, p<0.001; ANOVA; n=3/3/3 (2 exp.);

FIG. 7F: Representative confocal images of brain slices (hippocampus) from IMQ-treated Rag2^{-/-} and Rag2^{-/-}γc^{-/-} mice showing Evans Blue infiltration of brain parenchyma; n=3/3 (2 exp.);

FIG. 7G: Locomotor behavior of vehicle- vs. IMQ-treated Rag2^{-/-} and Rag2^{-/-}γc^{-/-} mice as measured by the open field test; no suppression was measured in Rag2^{-/-} mice; significant suppression of locomotion shown in Rag2^{-/-}γc^{-/-} mice is consistent with increased neuroinflammation also seen in Rag2^{-/-}γc^{-/-} as compared to Rag2^{-/-}mice; **** , p<0.0001, ANOVA; n=9/9 (2 exp); and

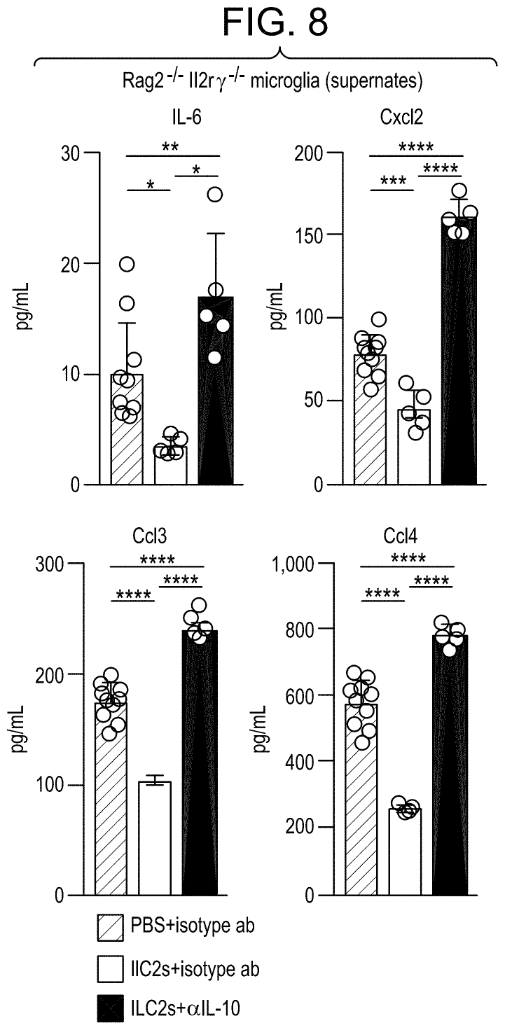

FIG. 8 shows blockade of IL-10 abolishes suppressive effects associated with ILC2 passive transfer: Luminex detection of secreted factors in supernates from microglia sorted from brains of mice transferred with PBS (control) or ILC2s with/without neutralizing antibodies to IL-10 and treated with 0.8 mg IMQ for 3 days prior to collection are shown; Student's T test; n=10/5/5 mice, results from two experiments combined are shown.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a”, “an”, and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human, to whom will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

In certain embodiments, the subject is in need of a treatment of a disorder related to microglial activation. Examples of such disorder include, but are not limited to: neurodegenerative disease, inflammatory disorder, neuropsychologic disorder, chronic pain, spinal cord injury, optic nerve inflammation, a viral or bacterial infection.

In certain embodiments, the subject is in need of a treatment of a neurodegenerative disease selected from the group consisting of Alzheimer's disease, Parkinson's disease, dementia, multiple sclerosis, a prion disease, amyotrophic lateral sclerosis, Huntington's disease, and aging.

In certain preferred embodiments, the subject is in need of a treatment of a neuropsychologic disorder selected from the group consisting of depression, anxiety, bipolar depression, and schizophrenia.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “in combination”, in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, the term “microglial activation” refers to a process associated with innate activation or adaptive activation of the microglia. Such activation can include morphological changes of the microglial cells, including shortening of cellular processes and enlargement of their soma, as well as the release of proinflammatory cytokines and chemokines, reactive oxygen and/or nitrogen intermediates, proteinases and complement proteins, and upregulation of cell surface activation antigens.

It has been demonstrated by the inventors for the first time that meningeal ILC2s exert potent regulatory influence at the borders of the brain through the secretion of IL-10. ILC-deficient mice displayed increased microglial reactivity at baseline, as well as exacerbated neuroinflammatory responses to either adaptive or innate immune challenge in models of EAE and IMQ-mediated systemic inflammation, respectively. In both of these models, ILC-deficient mice displayed increased brain infiltration of peripheral cells and compromised BBB integrity as shown by parenchymal leakage of molecules normally excluded from the brain.

Characterization of meningeal ILC subsets in mouse indicated ILC2s were predominant, and situated in sagittal sinuses thus with potential access to both CSF and blood; subsequent interrogation of human meninges samples suggested a similar population of ILC2s. RNAseq analysis of sorted meningeal ILC2s from control and IL-33 stimulated mice revealed an activation profile that mapped closely to canonical ILC2s by transcription factors and extracellular markers, but was unexpectedly dominated by an 1110 signature.

IL-10 production and release by IL-33 stimulated meningeal ILC2s at the level of protein was subsequently confirmed, as well as several other factors associated with resolution of inflammation and tissue protection, including Timp 1 , a factor shown to be protective at the BBB by suppression of Mmps, extracellular matrix and tight junction degrading peptidases. Accordingly, supernates from activated ILC2s were shown to robustly suppress cytokine, chemokine and Mmp release from microglia in vitro, with protective effects largely abolished when IL-10 signaling was blocked. In vivo, Rag2^{-/-}γc^{-/-} mice that received passive transfer of ILC2s showed meningeal ILC2 engraftment. Compared to PBS-injected controls, engrafted Rag2^{-/-}γc^{-/-} mice showed rectification of hyperactivated microglial phenotype at baseline and striking amelioration of neuroinflammation in the IMQ model. Critically, neuroprotection was abolished by neutralization of IL-10 or following transfer of IL-10^{-/-} ILC2s, indicating a key role for ILC2-derived IL-10.

Since their initial discovery, the diversity of ILC subtypes has grown substantially, with functional analogues to several T cell types having been well-characterized (Artis et al., Nature, 2015, 517: 293-301). Nevertheless, an ILC corollary to the T regulatory cell, i.e. an innate lymphoid regulatory cell, had remained conspicuously absent. Recently, however, a gut-resident lineage-negative cell lacking canonical ILC2 markers and with a transcription factor signature distinguished by Id3 vs. the expected Id2 common to ILCs, was designated as a source of protective IL-10 in a model of anti-CD40-induced colitis (Wang et al., Cell, 2017, 171(1):201-216). As such, this cell was suggested to be the long-enigmatic ‘ILC_{REG}’. It was further noted to be highly tissue-restricted. In a subsequent finding, a second specialized ILC eschewing production of IL-13 in favor of IL-10 —was revealed in lung and dubbed the ‘ILC2₁₀’ (Seehus et al., Nat Commun. 2017, 8(1):1900). Thus, tissue-restriction leaves global immunoregulatory function still unaddressed by the ILC family.

The transcriptional and functional analyses of meningeal ILC2s described in this application confirmed IL-10 as highly upregulated following IL-33 activation. Further detection of IL-10 production in canonical ILC2s from other murine tissues, and subsequently from human blood as described herein suggests that IL-10 production by ILC2s can be rule rather than exception. It is noted that Gata3, Irf4 and Nfil3, highlighted in the RNAseq dataset described herein, are bona-fide players in IL-10 production in other cell types, thus precluding a search for additional novel factors. Thus, ILC2s can unexpectedly represent a key innate lymphoid cell population with broad regulatory function. This previously unrecognized tool of classical ILC2s suggests ILC2 cell therapy as a novel therapeutic approach. Moreover, given the demonstrated importance of IL-33 to IL-10 production, the understanding of alarmins may require some reconsideration to include repair signaling—depending upon temporal or molecular context.

Observations described herein suggest that meningeal ILC2s are multipotent actors involved in neuroimmune homeostasis, brain-directed chemotaxis of peripheral immune cells by modulation of chemokines, e.g. CCL3 and CCL4, and further play an unanticipated role in BBB integrity, via both IL-10 and Timp1 and likely other mechanisms. In view of the present disclosure, additional factors/agents useful in regulation of neuroinflammation and barrier integrity can be identified. Such factors/agents may ultimately be useful—either through cell or small-molecule therapeutic approaches, in ameliorating CNS inflammatory pathologies such as multiple sclerosis, BBB disruption in meningitis or stroke, or psychiatric disorders co-morbid with chronic inflammation (Bhattacharya, et al., 2016, Psychopharmacology(Berl). 233:1623-36).

In a general aspect, the invention relates to methods of reducing or suppressing microglial activation or reducing BBB disruption in a subject in need thereof.

According to an embodiment of the present invention, the method comprises administering to the subject a therapeutically effective amount of type :II innate lymphoid cells (ILC2s), preferably activated ILC2s.

As used herein, the terms “type IT innate lymphoid cell,” “ILC2” and “ILC2 cell”, each refer to a population of lymphocytes that have a lymphoid morphology and an absence of T cell receptors, but can express type 2 helper T (Th2) cell-related cytokines upon activation. Examples of Th2 cytokines include interleukin 4 (IL-4), IL.-5, IL-9, IL-110 and IL-13. ILC2s are considered to be innate helper cells.

ILC2s can be identified by the expression of one or more markers using methods known in the art in view of the present disclosure. In some embodiments, the ILC2 cells are tested positive for expression of lymphoid markers CD90, tested positive for expression of lymphoid marker ICOS, and tested negative for expression of lineage markers associated with immune cell fate decision and maturation (referred to hereinafter as Lin). In some embodiments the ILC2 cells are tested positive for expression of lymphoid markers CD90 and ICOS, negative for expression of Lin, and further tested positive for expression of one or more additional markers of 1L7Ra (CD127), CD161., ST2, stern cell antigen 1 (Scal), IL2Ra (CD25), and CRT/12. For example, human ILC2 cells can be found in the gastrointestinal tract and lung and identified by their expression of CD161 and the diemoattractant receptor-homologous molecule expressed on Th2 cells (CRTH2) (Mjosberg et al., Nat Immunol. 2011; 12(11):1055-1062). ILC2s can be obtained according to known methods in view of the present disclosure. For example, cells can be isolated from bone marrow and enriched for ILC2s using commercially available kits and fluorescence activated cell sorting (FACS).

As used herein, the term “activated ILC2s” means ILC2s that have been contacted with an agent to induce production of Th2 cytokines by the ILC2s. In some embodiments the ILC2s are activated by a cytokine, such as IL-33, IL-25, IL-7, IL-2, and/or combinations thereof

Activated ILC2s can be obtained using methods known in the art in view of the present disclosure. The ILC2 cells can be isolated from various suitable sources of H,C2 cells including but not limited to lung tissue, gastrointestinal (GI) tract, CNS tissue, mammalian blood and/or blood products. In some embodiments, the ILC2 cells can be derived from human cord blood cells. Such human cord blood cells can be derived from a donor subject and/or from a patient's own cord blood. During the isolation, cells can be filtered through a Dacron mesh of a dimension corresponding to the cell of interest and then washed twice at 50×g for 1 min each. Cell viability can be determined by trypan blue dye exclusion. Cells with >90% viability can be used for transplantation. Ex vivo cells can be adult somatic cells, adult progenitor cells, adult stem cells, embryonic progenitor cells, or embryonic stem cells. Sources of such cells are well known to persons of ordinary skill in the art. Following isolation, the cells can be activated, optionally modified, ex vivo.

The ILC2 cells can be activated by co-culture with other cells and/or by culturing with one or more stimulatory or activation molecules, such as various cytokines. For example, like murine ILC2s, human ILC2s can be expanded and activated in vitro or ex vivo and produce significant quantities of IT,-5 and IL-13 in response to IL-2, IL-7 and. IL-33 or II-25. In some embodiments, activated ILC2 cells can be generated using in vitro methods, comprising collecting human cord blood from a subject, isolating c-Kit positive cells from the cord blood, and culturing the c-Kit positive cells in the presence of an IL-33 cytokine, whereby IL-33 activated ILC2 cells are generated. In some embodiments the ILC2 cells are activated by exposing the c-Kit positive cells in culture to one or more cytokines IL-33, IL-25, IL-2 and IL-7. In some embodiments the in vitro methods further comprise analyzing the generated ILC2 cells by measuring expression of one or more markers for ILC2, such as the expression of ST2, Killer cell lectin-like receptor subfamily G member 1 (KLRG1), SCA-1 or CD127 in the ILC2 cells. In some embodiments the activated ILC2 cells are substantially free of multipotent progenitor 2 (MPP2), which is IL-25 responsive but distinct from ILC2.

IL-33 is a cytokine belonging to the IL-1 superfamily. It is a dual-function protein that acts as a nuclear factor and pro-inflammatory cytokine. Nuclear localization and association with heterochromatin is mediated by the N-terminal domain and allows IL-33 to function as a novel transcriptional regulator of the p65 subunit of the NF-kappa B complex. The C-terminal domain is sufficient for binding to the ST2 receptor and activating the production of type 2 cytokines (e.g. IL-5 and IL-13) from polarized Th2 cells and ILC2 cells. Thus, in some embodiments activated ILC2 cells can be generated by contacting the ILC2 cells with IL-33 or its C-terminal domain.

The length of activation can be determined experimentally, which can depend on factors such as the origin of the ILC2s, the cytokine(s) and/or conditions used for activation, etc. In certain embodiments, the ILC2s are contacted with an agent, such as IL-33, IL-25, IL-2, IL-7, thymic stromal lymphopoietin (TSLP), for at least 30 minutes (e.g. at least 1 hour, 2 hours, 3 hours, 4 hours, 5 hours, 6 hours, 7 hours, or 8 hours). In preferred embodiments the ILC2s are contacted with a cytokine for at least 4 hours for activation prior to being administered to the subject.

In some embodiments, activated ILCs useful for the invention express CD90, SCA-1, iCOS and/or ST2, respond to IL-25 and/or IL-33, and generate IL-10, IL-5 and IL-13.

In certain embodiments of the application, the administered ILC2s are genetically modified. One skilled in the art would recognize that genetic modifications can be introduced into a cell utilizing methods known in the art in view of the present disclosure. For example, one or more viral vectors, or a viral vector and other gene delivering and editing tools, including the use of mRNA, siRNA, miRNA, or other genetic modifications, can be used in order to manipulate gene expression of any given relevant factor.

In certain embodiments, the ILC2s can be modified to express immune modulatory molecules including but not limited to cytokines, preferably, the ILC2s are modified to express IL-10. In certain embodiments, the ILC2s can be genetically modified to express an agent resulting in increased number of ILC2s and/or increased production of IL-10, preferably, the ILC2s are modified to express IL-33 receptor. In certain embodiments, the ILC2 cells can be genetically modified to express one or more other products of interest, such as one or more proteins known to be effective in reducing microglial activation, for example, TGF-beta.

As used herein, “autologous” refers to a biological matter or cells derived from tissues or cells of the subject or host. The activated ILC2s to be administered into a subject can be autologous.

As used herein, “heterologous” refers to a biological matter or cells derived from the tissues or cells of a different species or different individual of the same species as the subject or host (e.g., allogenic or xenogenic). The activated ILC2s to be administered into a subject can be heterologous.

In certain embodiments of the application, the method of reducing microglial activation or BBB disruption in a subject in need thereof, comprises administering to the subject a therapeutically effective amount of an agent capable of increasing the number of activated ILC2s in the subject. Examples of agents capable of increasing the number of activated ILC2s include, but are not limited to, agents capable of activating ILCs, such as IL-33, IL-25, thymic stromal lymphopoietin (TSLP), IL-2 and IL-7.

Increasing the number of activated ILC2 s in a subject can mean increasing the number of total activated ILC2s in the subject or increasing the number of specific tissue-resident activated ILC2s. ILC2s reside in the skin, lung, liver, gut, adipose, and brain of mammals. In preferred embodiments, the subject has an increased number of activated meningeal ILC2s as a result of one or more treatments of the invention.

In addition to agents known to be able to activate ILC2s and result in increased number of activated ILCs, other agents useful for reducing microglial activation or BBB disruption in a subject in need thereof can be identified using a method comprising:

(1) contacting ILC2s with a testing compound under a condition suitable for the growth and activation of the ILC2s; and

(2) measuring the number of activated ILC2s.

Conditions suitable for the growth and activation of the ILC2s are known to those skilled in the art. Suitable growth media for growing cells ex vivo are well known in the art and are disclosed for instance in “Culture of Animal Cells: A Manual of Basic Technique and Specialized Applications” R. I. Freshney, 2010, Wiley-Blackwell. The optimal medium for each type of cells can be obtained from specialized suppliers of the cells (e.g.: ATCC-LGC, MI, Italy; CDC, Atlanta, Ga., USA). The activated ILC2s can be identified and quantified by the expression of one or more markers for ILC2s, such as CD90 and ICOS, IL7Ra (CD127), CD161, ST2, stem cell antigen 1 (Scat), IL2Ra (CD25), and. CRTH2, and the negative for expression of Lin.

In certain other embodiments of the application, the method of reducing microglial activation in a subject in need thereof', comprises administering to the subject a therapeutically effective amount of an agent capable of increasing production of IL-10 by C2s in the subject.

Examples of agents capable of increasing the production of IL-10 by ILC2s include, but are not limited to IL-25, 1L-33, IL-2, IL-4, or a combination thereof Not all activated ILC2s have increased production of IL-10. The tissue location of the ILC2 seems to play a role. For example, ILC2s in lung produce very little if any IL-10 following treatment with IL-33 alone. However, if ILC2s in lung are excited by IL-33 and IL-2 and/or IL-4, they produce high levels of IL-10. In certain embodiment, combinations of cytokines can be used to obtain synergistic response in increased production of IL-10 by ILC2s. For example, the combination of IL-25 and IL-33 can lead to much higher IL-10 production than each separately. In certain other embodiment, combinations of cytokines can be used to obtain synergistic response in increased production of Timp1 by ILC2s.

Increasing the production of IL-10 or Timp1 by activated ILC2s in a subject can mean increasing the production of IL-10 or Timp1 by the total activated ILC2s in the subject or increasing the production of IL-10 or Timp1 by specific tissue-resident activated ILC2s. In preferred embodiments, the subject has an increased production of IL-10 or Timp1 by activated meningeal ILC2s as a result of one or more treatments of the invention.

In addition to agents known to be able to increase production of IL-40 or Timp1 by ILC2s, other agents useful for reducing microglial activation in a subject in need thereof can be identified using a method comprising:

(1) contacting ILC2s with a testing compound under a condition suitable for the production of IL-10 by the ILC2s; and

(2) measuring a level of IL-10 or Timp1 produced by the ILC2s.

Conditions suitable for the production of IL-10 by the ILC2s are known to those skilled in the art. The IL-10 or Timp1 produced by the ILC2s can be identified and quantified using methods known in the art in view of the present disclosure, e.g., by an antibody specific to IL-10 or Timp1.

In certain aspects, the invention relates to a pharmaceutical composition for reducing microglial activation in a subject in need thereof.

in one embodiment, the pharmaceutical composition comprises a therapeutically effective amount of activated ILC2s and a pharmaceutically acceptable carrier.

In another embodiment, the pharmaceutical composition comprises a therapeutically effective amount of an agent capable of increasing the number of activated type II innate lymphoid cells (ILC2s) in the subject and a pharmaceutically acceptable carrier,

In yet another embodiment, the pharmaceutical composition comprises a therapeutically effective amount of an agent capable of increasing production of IL-10 or Timp1 by an activated ILC2 in the subject and a pharmaceutically acceptable carrier. The production of Timp1 can be increased directly by the activated ILC2, or indirectly by another factor (e.g., IL-10) whose production and/or activity is increased by the activated ILC2.

A pharmaceutically acceptable carrier is non-toxic and should not interfere with the efficacy of the active ingredient. Pharmaceutically acceptable carriers can include, but are not limited to, one or more, such as water, glycols, sugar, oils, amino acids, alcohols, preservatives, emollients, stabilizers, coloring agents and the like. Any suitable pharmaceutically acceptable carrier can be used together with activated ILC2s, an agent that increases the number of ILC2s, or an agent increasing production of IL-10 or Timp1, for administration to a subject. For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, mannitol or another sugar, and phosphate-buffered saline (PBS).

It should be understood that in addition to the ingredients particularly mentioned above the formulations of this presently disclosed subject matter can include other agents conventional in the art having regard to the type of formulation in question. For example, sterile pyrogen-free aqueous and non-aqueous solutions can be used.

The therapeutic regimens and compositions of the presently disclosed subject matter can be used with additional agents or biological response modifiers including, but not limited to, the cytokines.

Administration of the compositions of the presently disclosed subject matter can be by any method known to one of ordinary skill in the art, including, but not limited to intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation. in some embodiments, suitable methods for administration of a composition of the presently disclosed subject matter include, but are not limited to, intravenous injection. Alternatively, a composition can be deposited at a site in need of treatment in any other manner. The particular mode of administering a composition of the presently disclosed subject matter depends on various factors, including the distribution and abundance of cells to be treated, additional tissue-or cell-targeting features of the composition, and mechanisms for metabolism or removal of the composition from its site of administration.

According to embodiments of the invention, administration of isolated ILC2s or a pharmaceutical composition thereof can be systemic or local. In certain embodiments, administration is parenteral. In preferred embodiments, administration of isolated ILC2s or a pharmaceutical composition thereof to a subject is by injection, infusion, intravenous (IV) administration, intrathecal administration, or intrafemoral administration. In still further preferred embodiments, administration of isolated ILC2s or a pharmaceutical composition thereof to a subject is by intravenous or intrathecal administration.

Administration of an agent, such as a cytokine, that increases the number of activated ILC2s or the production of IL-10 by ILC2s, can be intramuscular, subcutaneous, or intravenous. However other modes of administration such as cutaneous, intradermal or nasal can be envisaged as well. Intramuscular administration of the agent can be achieved by using a needle to inject a suspension of the agent composition. An alternative is the use of a needleless injection device to administer the composition (using, e.g., Biojector™) or a freeze-dried powder of the agent composition.

In certain embodiments, ILC2s can be harvested from a patient, optionally genetically modified, activated by ex vivo treatment, and then administered back into the patient to reduce microglial activation.

For intravenous, cutaneous or subcutaneous injection, the agent composition can be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

An effective dose of a composition of the presently disclosed composition is administered to a subject in need thereof. As used herein, an “effective amount” refers to an amount of a composition which, upon administration to a subject in need thereof, provides a desired local or systemic effect in the subject. In some embodiments, an effective amount is an amount sufficient to effectuate a beneficial or desired clinical result of reduced microglial activation in the subject. The effective amounts can be provided all at once in a single administration or in fractional amounts that provide the effective amount in several administrations.

Actual dosage levels of active ingredients in the compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, and factors individual to each subject, including their size, age, injury, and/or the disease condition and prior medical history, and amount of time since the disease occurred or the disease began. However, it is within the skill of the art to start doses of the compositions at levels lower than required to achieve the desired therapeutic effect and to gradually increase the dosage until the desired effect is achieved in view of the present disclosure.

For example, after review of the disclosure of the presently disclosed subject matter presented herein, one of ordinary skill in the art can tailor the dosages to an individual patient, taking into account the particular formulation, method for administration to be used with the composition, and severity of the condition. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

The contents of all cited references (including literature references, issued patents, published patent applications, and co-pending patent applications) cited throughout this application are hereby expressly incorporated by reference.

### EMBODIMENTS

The invention provides also the following non-limiting embodiments.

Embodiment 1 is a method of reducing or suppressing microglial activation in a subject in need thereof, comprising administering to the subject a therapeutically effective amount of activated type II innate lymphoid cells (ILC2s).

Embodiment 1a is a method of reducing blood-brain barrier (BBB) permeability in a subject in need thereof, comprising administering to the subject a therapeutically effective amount of activated type II innate lymphoid cells (ILC2s).

Embodiment 2 is the method of embodiment 1 or 1a, wherein the ILC2 is activated by contacting the ILC2 with at least one cytokine selected from the group consisting of IL-33, IL-25, IL-2, IL-7, or a combination thereof.

Embodiment 2a is the method of embodiment 2, wherein the ILC2 is activated by contacting the ILC2 with two cytokines selected from the group consisting of IL-33, IL-25, IL-2, IL-7.

Embodiment 2b is the method of embodiment 2, wherein the ILC2 is activated by contacting the ILC2 with three cytokines selected from the group consisting of IL-33, IL-25, IL-2, IL-7.

Embodiment 2c is the method of embodiment 2, wherein the ILC2 is activated by contacting the ILC2 with the cytokines of IL-33, IL-25, IL-2 and IL-7.

Embodiment 2d is the method of embodiment 2, wherein the ILC2 is activated by contacting the ILC2 with IL-33 and IL-25.

Embodiment 2e is the method of any one of embodiments 2 to 2d, wherein the ILC2 is activated by contacting the ILC2 with the at least one cytokine for at least 30 minutes, preferably at least 2 hours, more preferably at least 4 hours.

Embodiment 3 is the method of any one of embodiments 1 to 2d, wherein the administered activated ILC2 cells are autologous.

Embodiment 3(a) is the method of any one of embodiments 1 to 2d, wherein the administered activated ILC2 cells are allogeneic.

Embodiment 3(b) is the method of any one of embodiments 1 to 2d, wherein the administered activated ILC2 cells are syngeneic.

Embodiment 4 is the method of any one of embodiments 1 to 3(b), wherein the ILC2s are genetically modified.

Embodiment 4a is the method of embodiment 4, wherein the cells are genetically modified for increased production compared to otherwise identical unmodified cells.

Embodiment 4b is the method of embodiment 4, wherein the cells are genetically modified for increased number of activated ILC2s compared to otherwise identical unmodified cells.

Embodiment 4c is the method of embodiment 4, wherein the cells are genetically modified for increased Timp1 production compared to otherwise identical unmodified cells.

Embodiment 5 is the method of any one of embodiments 1 to 4c, wherein the therapeutically effective amount of activated ILC2s is administered intravenously or intrathecally.

Embodiment 6 is a method of reducing microglial activation in a subject in need thereof comprising administering to the subject a therapeutically effective amount of an agent capable of increasing the number of activated type II innate lymphoid cells (ILC2s) in the subject.

Embodiment 6a is a method of reducing blood-brain barrier (BBB) permeability in a subject in need thereof, comprising administering to the subject a therapeutically effective amount of an agent capable of increasing the number of activated type II innate lymphoid cells (ILC2s) in the subject.

Embodiment 7 is the method of embodiment 6 or 6a, comprising administering to the subject at least one cytokine selected from the group consisting of IL-33, IL-25, IL-2, IL-7, or a combination thereof.

Embodiment 7a is the method of embodiment 7, comprising administering to the subject two cytokines selected from the group consisting of IL-33, IL-25, IL-2, IL-7.

Embodiment 7b is the method of embodiment 7, comprising administering to the subject three cytokines selected from the group consisting of IL-33, IL-25, IL-2, IL-7.

Embodiment 7c is the method of embodiment 7, comprising administering to the subject IL-33, IL-25, IL-2, IL-7.

Embodiment 8 is a method of reducing microglial activation in a subject in need thereof, comprising administering to the subject an effective amount of an agent capable of increasing production of IL-10 by an activated type II innate lymphoid cell (ILC2) in the subject.

Embodiment 8a is a method of reducing microglial activation in a subject in need thereof, comprising administering to the subject an effective amount of an agent capable of increasing production of Timp 1 by an activated type II innate lymphoid cell (ILC2) in the subject.

Embodiment 8b is a method of reducing blood-brain barrier (BBB) permeability in a subject in need thereof, comprising administering to the subject an effective amount of an agent capable of increasing production of IL-10 by an activated type II innate lymphoid cell (ILC2) in the subject.

Embodiment 8c is a method of reducing blood-brain barrier (BBB) permeability in a subject in need thereof, comprising administering to the subject an effective amount of an agent capable of increasing production of Timp 1 by an activated type II innate lymphoid cell (ILC2) in the subject.

Embodiment 9 is the method of any one of embodiments 8 to 8c, comprising administering to the subject a therapeutically effective amount of IL-33, IL-25, IL-2, IL-4, or a combination thereof.

Embodiment 10 is the method of any one of embodiments 1 to 9, herein the subject is in need of a treatment of a disorder related to microglial activation.

Embodiment 11 is the method of embodiment 10, wherein the subject is in need of a treatment of a neurodegenerative disease, inflammatory disorder, neuropsychologic disorder, chronic pain, traumatic brain injury, spinal cord injury, optic nerve inflammation, a viral or bacterial infection.

Embodiment 12 is the method of embodiment 10, wherein the subject is in need of a treatment of a neurodegenerative disease selected from the group consisting of Alzheimer's disease, Parkinson's disease, dementia, multiple sclerosis, a prion disease, amyotrophic lateral sclerosis, Huntington's disease, aging, meningitis and stroke.

Embodiment 13 is the method of embodiment 10, wherein the subject is in need of a treatment of a neuropsychologic disorder selected from the group consisting of depression, anxiety, bipolar depression, and schizophrenia.

Embodiment 14 is a pharmaceutical composition for reducing microglial activation in a subject in need thereof, comprising a therapeutically effective amount of isolated activated type II innate lymphoid cells (ILC2s) and a pharmaceutically acceptable carrier.

Embodiment 14a is the pharmaceutical composition of embodiment 14, further comprising at least one agent capable of activating ILC2s or maintaining the ILC2s in activated state in the composition.

Embodiment 14b is the pharmaceutical composition of embodiment 14a, wherein the at least one agent is selected from the group consisting of IL-33, IL-25, IL-2, and IL-7.

Embodiment 14c is the pharmaceutical composition of embodiment 14a, wherein the at least one agent comprises two agents selected from the group consisting of IL-33, IL-25, IL-2, and IL-7.

Embodiment 14d is the pharmaceutical composition of embodiment 14a, wherein the at least one agent comprises three agents selected from the group consisting of IL-33, IL-25, IL-2, and IL-7.

Embodiment 14e is the pharmaceutical composition of embodiment 14a, wherein the at least one agent comprises IL-33, IL-25, IL-2, and IL-7.

Embodiment 14f is the pharmaceutical composition of embodiment 14a, wherein the at least one agent comprises IL-33 and IL-25.

Embodiment 14g is the pharmaceutical composition of any one of embodiments 14 to 14f, wherein the ILC2s are meningeal ILC2s.

Embodiment 15 is a pharmaceutical composition for reducing microglial activation in a subject in need thereof, comprising a therapeutically effective amount of an agent capable of increasing the number of activated type 11 innate lymphoid cells (ILC2s) in the subject and a pharmaceutically acceptable carrier.

Embodiment 15a is the pharmaceutical composition of embodiment 15, wherein the pharmaceutical composition comprises at least one agent selected from the group consisting of IL-33, IL-25, thymic stromal lymphopoietin (TSLP), IL-2, and IL-7.

Embodiment 15b is the pharmaceutical composition of embodiment 15, wherein the pharmaceutical composition comprises two agents selected from the group consisting of IL-33, IL-25, thymic stromal lymphopoietin (TSLP), IL-2, and IL-7.

Embodiment 15c is the pharmaceutical composition of embodiment 15, wherein the pharmaceutical composition comprises three agents selected from the group consisting of IL-33, IL-25, thymic stromal lymphopoietin (TSLP), IL-2, and IL-7.

Embodiment 15d is the pharmaceutical composition of embodiment 15, wherein the pharmaceutical composition comprises four agents selected from the group consisting of IL-33, IL-25, thymic stromal lymphopoietin (TSLP), IL-2, and IL-7.

Embodiment 15e is the pharmaceutical composition of embodiment 15, wherein the pharmaceutical composition comprises IL-33, IL-25, thymic stromal lymphopoietin (TSLP), IL-2, and IL-7.

Embodiment 15f is the pharmaceutical composition of embodiment 15, wherein the pharmaceutical composition comprises IL-33 and IL-25.

Embodiment 15g is the pharmaceutical composition of any one of embodiments 15 to 15f, wherein the ILC2s are meningeal ILC2s.

Embodiment 16 is a pharmaceutical composition for reducing microglial activation in a subject in need thereof, comprising a therapeutically effective amount of an agent capable of increasing production of IL-10 by an activated type II innate lymphoid cell (ILC2) in the subject and a pharmaceutically acceptable carrier.

Embodiment 16a is the pharmaceutical composition of embodiment 16, wherein the pharmaceutical composition comprises at least one agent selected from the group consisting of IL-33, IL-25, thymic strormal lymphopoietin (TSLP), IL-2, and IL-7.

Embodiment 16b is the pharmaceutical composition of embodiment 16, wherein the pharmaceutical composition comprises two agents selected from the group consisting of IL-33, IL-25, thymic stromal lymphopoietin (TSLP), IL-2, and IL-7.

Embodiment 16c is the pharmaceutical composition of embodiment 16, wherein the pharmaceutical composition comprises three agents selected from the group consisting of IL-33, IL-25, thymic stromal lymphopoietin (TSLP), IL-2, and IL-7.

Embodiment 16d is the pharmaceutical composition of embodiment 16, wherein the pharmaceutical composition comprises four agents selected from the group consisting of IL-33, IL-25, thymic stromal lymphopoietin (TSLP), IL-2, and IL-7.

Embodiment 16e is the pharmaceutical composition of embodiment 16, wherein the pharmaceutical composition comprises IL-33, IL-25, thymic stromal lymphopoietin (TSLP), IL-2, and IL-7.

Embodiment 16f is the pharmaceutical composition of embodiment 16, wherein the pharmaceutical composition comprises IL-33 and IL-25.

Embodiment 16g is the pharmaceutical composition of any one of embodiments 16 to 16f, wherein the ILC2s are meningeal ILC2s.

Embodiment 17 is a method of preparing the pharmaceutical composition of any one of embodiments 14 to 14g, comprising mixing the therapeutically effective amount of isolated activated type II innate lymphoid cells (ILC2s) with the pharmaceutically acceptable carrier.

Embodiment 18 is a method of preparing the pharmaceutical composition of any one of embodiments 15 to 15g, comprising mixing the therapeutically effective amount of the agent capable of increasing the number of ILC2s in the subject with the pharmaceutically acceptable carrier.

Embodiment 19 is a method of preparing the pharmaceutical composition of any one of embodiments 16 to 16g, comprising mixing the therapeutically effective amount of the agent capable of increasing production of IL-10 by the activated type 11 innate lymphoid cell (ILC2) in the subject with the pharmaceutically acceptable carrier.

Embodiment 20 is a method of identifying an agent useful for reducing microglial activation in a subject in need thereof, comprising:

(1) contacting an innate lymphoid cell (ILC2) with the agent under a condition suitable for the growth of the ILC2; and

(2) measuring the number of the ILC2s; wherein an increased number of the ILC2s compared to a control level is indicative the agent useful for reducing microglial activation in a subject in need thereof.

Embodiment 21 is a method of identifying an agent useful for reducing microglial activation in a subject in need thereof, comprising:

(1) contacting an innate lymphoid cell (ILC2) with the agent; and

(2) measuring a level of IL-10 produced by the ILC2;

wherein an increased amount of IL-10 produced by the ILC2 compared to a control level is indicative the agent useful for reducing microglial activation in a subject in need thereof.

Embodiment 21a is a method of identifying an agent useful for reducing microglial activation in a subject in need thereof, comprising:

(1) contacting an innate lymphoid cell (ILC2) with the agent; and

(2) measuring a level of Timp1 produced by the ILC2;

wherein an increased amount of Timp1 produced by the ILC2 compared to a control level is indicative the agent useful for reducing microglial activation in a subject in need thereof

Embodiment 21b is a method of identifying an agent useful for reducing BBB permeability in a subject in need thereof, comprising:

(1) contacting an innate lymphoid cell (ILC2) with the agent; and

(2) measuring a level of IL-10 produced by the ILC2;

wherein an increased amount of IL-10 produced by the ILC2 compared to a control level is indicative the agent useful for reducing BBB permeability in a subject in need thereof.

Embodiment 21c is a method of identifying an agent useful for reducing BBB permeability in a subject in need thereof, comprising:

(1) contacting an innate lymphoid cell (ILC2) with the agent; and

(2) measuring a level of Timp1 produced by the ILC2;

wherein an increased amount of Timp1 produced by the ILC2 compared to a control level is indicative the agent useful for reducing BBB permeability in a subject in need thereof.

Embodiment 22 is the method of any one of embodiments 1 to 13, the pharmaceutical composition of any one of embodiments 14-16, or the method of any one of embodiments 17-21c, wherein the ILC2s express one or more of CD90, ICOS, IL7Ra (CD127), CD161, ST2, stern cell antigen 1 (Sca1), IL2Ra (CD25), and CRTH2, and are negative for expression of Lin.

Embodiment 22a is the method of embodiment 22, wherein the ILC2s expresses two of CD90, ICOS, IL7Ra (CD127), CD161, ST2, stem cell antigen 1 (Sca1t), IL2Ra (CD25), and CRTH2, and is negative for expression of Lin.

Embodiment 22b is the method of embodiment 22, wherein the ILC2s expresses three of CD90, ICOS, IL7Ra (CD127), CD161, ST2, stern cell antigen 1 (Sca1), IL2Ra (CD25), and CRTH2, and is negative for expression of Lin.

Embodiment 22c is the method of embodiment 22, wherein the ILC2s expresses four of CD90, ICOS, IL7Ra (CD127), CD161, ST2, stem cell antigen 1 (Sca1), IL2Ra (CD25), and CRTH2, and is negative for expression of Lin.

Embodiment 22d is the method of embodiment 22, wherein the ILC2s expresses five of CD90, ICOS, IL7Ra (CD127), CD161, ST2, stern cell antigen 1 (Sca1), IL2Ra, (CD25), and CRTH2, and is negative for expression of Lin.

Embodiment 22e is the method of embodiment 22, wherein the ILC2s expresses six of CD90, ICOS, IL7Ra (CD127), CD161, ST2, stem cell antigen 1 (Sca1), IL2Ra (CD25), and CRTH2, and is negative for expression of Lin.

Embodiment 22f is the method of embodiment 22, wherein the ILC2s expresses seven of CD90, ICOS, IL7Ra (CD127), CD161, ST2, stem cell antigen 1 (Sca1), IL2Ra (CD25), and CRTH2, and is negative for expression of Lin.

Embodiment 22g is the method of embodiment 22, wherein the ILC2s expresses eight of CD90, ICOS, IL7Ra (CD127), CD161, ST2, stern cell antigen 1 (Sca1), IL2Ra, (CD25), and CRTH2, and is negative for expression of Lin.

Embodiment 22h is the method of any one of embodiments 22 to 22 g, wherein the activated ILC2s produces IL-10.

Embodiment 22i is the method of any one of embodiments 22 to 22h, wherein the activated ILC2s further produces at least one of 1L-4, 11 -5, 1L-9 and IL-13

Embodiment 22j is the method of embodiment 22i, wherein the activated ILC2s further produces two of 1L-4, 1L-5, 1L-9 and 1L-13.

Embodiment 22 k is the method of embodiment 22j, wherein the activated ILC2s further produces three of1L-4, 11,5, IL-9 and 1L-13,

Embodiment 221 is the method of embodiment 22j, wherein the activated ILC2s further produces IL-4, 1L-5, 11,9 and 1L-13.

Embodiment 22m is the method of any one of embodiments 22 to 221, wherein the activated ILC2s produces Timp1.

Embodiment 22n is the method of any one of embodiments 1 to 22m, wherein the activated ILC2s are meningeal ILC2s.

### EXAMPLES

The following Examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter. The examples do not limit the invention in any way. They merely serve to clarify the invention.

### Example 1. ILC2s are Enriched in Meninges

Transcription factor labeling of CD45+Lin−FCϵr1α−DX5−CD90+IL7rα+cells (FIG. 1A) was used to resolve ILC1 (Tbet+) ILC2 (Gata3Hi) and ILC3 (RORγt+Gata3−/lo) subsets. First, meningeal innate lymphoid cells (mILCs) were profiled to determine the most abundant type. Brains of nave mice were carefully dissected and placed immediately into 4% paraformaldehyde for fixation. After 48 hours (hr) of fixation at 4° C., brains were incubated for 24 hrs in 30% sucrose in H₂O and subsequently stored at −80° C. Skullcaps were placed in 20 ml fluorescence activated cell sorting (FACS) buffer (DMEM/F-12 +2% FBS) in petri dishes and meninges peeled carefully away using forceps. Meninges were placed in a 70 μm cell filter and gently dissociated using the reverse side of a syringe plunger with (minimum 100) circular strokes. Cells were then washed through the filter three times to further release cells from dissociated tissue. Tubes were spun using a centrifuge for 7 minutes (min), 1500 RPM at 4° C. and then carefully decanted. Pellets were resuspended in 200 ul FACS buffer and cells placed on ice. Cells were labeled extracellularly with antibodies to Lineage markers, FCϵrlα, DX5, CD45, CD90, IL7rα, fixed, permeabilized and subsequently labeled intranuclearly for TBET, GATA3, and RoRγt using the TrueNuclear® labeling kit (Biolegend) as per manufacterer's protocol. Transcription factor FACS revealed ILC2 (ILC2) were the predominant population in dural lymphatics in mice (FIG. 1B).

To explore possible anatomical routes by which ILC2s could feasibly influence the CNS, immunolabeling and high-resolution confocal imaging of intact meninges were conducted using CD90 to label ILCs and Lyvel to delineate lymphatic vs. non-lymphatic tissues (Louveau et al., 2015, Nature 523, 337-341). CD90+0 ILCs were visualized predominantly within sinuses but were not confined to Lyvel+lymphatics per se (FIG. 1C(i)); the addition of in situ Gata3 immunolabeling confirmed sinus presence of ILC2s (FIG. 1C(ii)). As conduits for both cerebrospinal fluid (CSF) and CNS blood flow, the sinuses suggested secreted factors as a possible mechanistic link allowing meningeal ILCs to interact with other cells in CNS.

ILCs had not previously been characterized (or even identified) in human meninges. Their presence in fresh post-mortem meninges was investigated in this study. Briefly, upon receipt, 18-24 hours post-mortem sagittal sinus, pial and choroid plexus tissues were dissected, carefully PBS-washed, dissociated, and labeled for analysis by flow cytometry. CD45+Lin−CD11c−FcϵR1−CD127+CRTH2+CD161+cells were successfully resolved in all samples (FIG. 1D). Early results from the studies with fresh post-mortem meninges suggest that human meninges also harbor an analogous population of ILC2s as that found in mouse meninges.

### Example 2. IL-33 and IL-25 Activates Meningeal ILC2

To probe relative functional response of IL-25 and IL-33, Rag2^{-/-} mice were treated with each cytokine by intraperitoneal (i.p.) injection; mice were injected with 0.033 mg/kg, daily, for 3 days 24 hours apart. Brains and meninges were removed as described in Example 1. Cells were isolated, labeled with antibodies to Lineage markers, FCϵrlα, DX5, CD45, CD90, IL7rα, KLRG1, ST2, GATA3, KI-67, and analyzed with FACS. Increased proliferation as measured by mILC2 percentage of CD45+, counts, and K1-67 labeling, was seen with injection of either factor, but most robustly with IL-33.

### Example 3. ILC2-Deficient Mice Exhibit Microglial Activation

Microglia, the principal immune presence within CNS, are engaged in constant surveillance and respond readily to protect delicate surrounding neural tissues. To examine the role of mILC2-derived factors in CNS immunomodulation, microglia were isolated from brains of ILC2-deficient Rag2^{-/-} γ^{-/-} mice and compared to those from control Rag2^{-/-} mice using FACS.

Immunolabeling with Iba-1 indeed suggested visible disparities in microglial density (FIG. 3A) with brains from ILC-deficient mice showing increased numbers of microglia per unit area (FIG. 3B). Subsequent analysis of acutely-isolated brain cell suspensions by flow cytometry revealed elevated side scatter (a general indication of cell granularity thus implying intracellular activity) (FIG. 3C), increased expression of CD45 (FIG. 3D) and other surface activation markers, e.g., FcRII/III and MARCO (FIG. 3E) by microglia from ILC-deficient mice. These observations indeed suggested an altered baseline microglial state associated with ILC deficiency. Activation markers associated with both type-I and type-II responses were elevated similarly, suggesting general activation rather than M1- or M2-skewing.

In order to probe the functional role of mILC2, control and ILC2-deficient mice were injected with 0.033 mg/kg, daily, for 3 days 24 hours apart with rIL-33 to activate ILC2s. Then meninges and brain cells were isolated and placed together in culture at 37° C. in RPMI with 10% FBS, 1:100 P/S, 1:100 L-glutamine, 1:100 NEAA, 1:100 Na Pyruvate, 1:1000 Beta-mercaptoethanol (All Gibco). IL-33 treatment augmented differences between ILC2-sufficient and -deficient mice, with secretory profiles, as analyzed by Luminex bead assay showing increased levels of several cytokines and chemokines in ILC2-deficient mice.

This “hyperactivated” microglial state in ILC2-deficient mice could be explained by a loss of immunosuppressive influence by ILCs, which could feasibly potentiate disinhibition of microglia. Alternatively, however, a leaky blood-brain and/or blood-CSF barrier (BBB, BCB) could also lead to increases in parenchymal levels of normally peripherally-restricted immunostimulatory factors and result in “hyperactivation” of microglia. The first scenario would be consistent with a direct immunoregulatory role for ILCs, and the second with the alternative possibility that, similar to gut ILCs, meningeal ILCs might be involved in regulating barrier function. In order to comprehensively interrogate these possibilities, responses to both adaptive and innate immune challenge were characterized. Experimental autoimmune encephalomyelitis (EAE) involving direct autoimmune attack of the CNS, was selected as an adaptive model, whereas topical administration of Imiquimod (IMQ), an immunostimulant shown to induce both skin and brain inflammation, was chosen to examine interactions between innate effectors at the borders of the brain, including ILCs and microglia.

It was found that increased EAE severity in ILC-deficient mice parallels a failure of T cells to arrest in meninges. In brief, CD4+ T cells sorted from Mog₃₅₋₅₅ peptide-immunized wild type mice were transferred i.v. to Rag2^{-/-} and Rag2^{-/-} γc^{-/-} mice. Passive EAE induction ensured equivalent numbers and pathogenicity of T cells between groups. Notably, the initial experiment required premature termination as all but one Rag2^{-/-} γc^{-/-} mice progressed rapidly to complete paralysis and/or moribund status within 24-36 h of detection of first symptoms, whereas Rag2^{-/-} mice displayed expected progression. Therefore, CD4 numbers were titrated by 50% allowing an attenuated disease course wherein ILC-deficient mice still showed initial symptoms slightly earlier than controls and subsequently elevated disease severity but with reduced mortality (FIG. 4A). Along with severity, incidence of EAE was markedly increased in Rag2^{-/-} γc^{-/-} mice (FIG. 4B). In line with higher clinical scores, ILC-deficient mice showed greater T cell infiltration of parenchyma (FIG. 4C i-ii). Interestingly, however, Rag2^{-/-} meninges contained significantly more T cells than Rag2^{-/-} γc^{-/-} meninges (FIG. 4D i-ii), the reverse of what was observed in brain and spinal cord. Quantification of serum factors showed either no difference or a trend towards increased pro-inflammatory cytokines in Rag2^{-/-} mice, arguing against the hypothesis that T cells in circulation were rendered less pathogenic when in the presence of peripheral ILCs in lymph nodes or spleen.

This inverse relationship between parenchymal and meningeal accumulation of T cells in Rag2^{-/-} and Rag2^{-/-} γc^{-/-} CNS suggested that ILCs were potentially involved in arresting BBB crossing of encephalitogenic T cells.

Additionally, ILC-deficient mice showed decoupled skin vs. brain responses to topical IMQ. The TLR7/8 agonist IMQ, widely-used topically to induce psoriatic skin inflammation in rodent models, was also shown to promote vigorous microglial response and immune infiltration of brain. Coupled with the finding that Rag2^{-/-} mice retain moderate psoriatic response to IMQ, yet Rag2^{-/-} γc^{-/-} do not, suggested this to represent a unique model by which to simultaneously probe microglia, ILCs and barrier integrity. It should be noted that models of peripheral inflammation may have relevance to CNS pathologies, given well-documented clinical comorbidities of psychiatric disorders with peripheral inflammatory disease, including mood disorders with psoriasis.

Skin and brain responses to IMQ by wild type, Rag2^{-/-} and Rag2^{-/-} γc^{-/-} mice were compared. As expected, clinical scoring (FIG. 7A) and histology (FIG. 7B) of skin showed severe, moderate, and little-to-no pathology in wild type, Rag2^{-/-} and Rag2^{-/-} γc^{-/-} mice, respectively (FIG. 7B). Surprisingly, however, hematoxylin and eosin staining (H&E) performed on brains from the same groups revealed micro-hemorrhaging in a different pattern—i.e. equivalently mild in both wild type and Rag2^{-/-} mice but unexpectedly severe in Rag2^{-/-} γc^{-/-} mice (FIG. 7C). Flow cytometric analysis of dissociated brains showed an analogous pattern of monocyte infiltration (FIG. 7D) wherein brains from ILC-deficient mice showed significantly more monocytes (FIG. 7E). Therefore, ILCs—yet surprisingly not T cells—appeared to be pivotal to the observed phenotype of BBB disruption.

Although parenchymal micro-hemorrhages and immune infiltration could be considered valid measures of integrity on a macroscopic level, changes in BBB permeability to subcellular particles were also assessed. IMQ treatment was monitored with transcardial injection of Evans Blue dye, which is normally excluded from brain by intact blood-brain and blood-CSF barriers (BCB). In concordance with measures of cell infiltration, brains from ILC-deficient mice showed increased levels of parenchymal Evans Blue staining compared to Rag2^{-/-} controls (FIG. 7F).

### Example 4. ILC2 Transfer Suppresses Microglial Activation

To probe ILC2-specific effects on microglia, passive intravenous transfer of ILC2s or PBS (control) into Rag2^{-/-} γc^{-/-} or Rag2^{-/-} control mice was performed.

### ILC2 Isolation

Rag2^{-/-} mice were intraperitoneally (i.p.) injected with 0.033 mg/kg recombinant IL-33 daily for three days. Mice were euthanized and tibia and femurs were isolated in a sterile hood. Bones were cleaned of muscle with forceps and then kept on ice in sterile PBS. Bones were gently crushed, four at a time, in sterile PBS using mortar and pestle. Released bone marrow cells and all bone matter were transferred via pipetman into 50 ml conical tubes, passed through a 70 μm cell filter to filter out large particles. Cells were then passed through a 40 μm cell filter to further remove any debris. Tubes were spun using a centrifuge for 10 min at 1500 RPM in 4° C. and then decanted. Pellets were resuspended in red blood cell lysis buffer and incubated at room temperature for 2 min. Ice-cold PBS was added to dilute and stop cell lysis. Tubes were spun using a centrifuge for 10 min at 1500 RPM in 4° C. and then decanted. Pellets were resuspended in 2% BSA, viable cells were counted, and cell concentrations were adjusted to 1×10⁸/m1 and cells placed on ice. ILC2 cells were enriched using EasyStep™ Mouse ILC2 Erichment Kit according to manufacturer's instructions.

### ILC2 Sorting

Enriched cells were labeled using the following cocktail of antibodies at titration of 2 ul/10⁶ cells with the exception of Live/Dead, used at 1 ul/10⁶ cells and Lineage cocktail, used at 20 ul/10̂6 cells: Zombie Aqua Live/Dead, Lineage cocktail, +FCϵrl a, +CD49b, CD45, CD90.2, IL7rα, ST2. Live, single, CD45+Lineage-CD49b-FCϵr1a-CD90.2+IL7rα+ST2+ events were sorted into ILC2 media.

### ILC2 Expansion

Sorted cells were placed at a density of 1×10⁶/ml in 100 ul ILC2 media supplemented with recombinant IL-2 at 50 units/ml, and recombinant IL-7 at 50 ng/ml in 96-well TC-coated round-bottom plates. Cells were expanded for 2 days in these plates and then moved to 48-well TC-coated flat-bottom plates for 3 additional days. On day 5, 100 ng/ml rIL-33 (final concentration) was added. On Day 6, cells were collected, washed twice in sterile PBS and resuspended in sterile saline at 5×10⁵/ml.

### ILC2 Passive transfer

Female Rag2^{-/-}γc^{-/-} mice were injected with 100 uL of ILC2 cell suspension (0.5×10⁶ cells) or equal volume saline (controls) in the tail vein. On day 41, mice were euthanized via CO₂ inhalation. Euthanized mice were immediately perfused transcardially with Perfusion Buffer for 3 min on ‘fast’ at speed 4.5 using a Variable Speed Pump (Fisher Scientific, Cat# 13-876-1) to remove all blood. Bone marrow was isolated as described above. Meninges single cells were isolated as described in Example 1. Cells were then analyzed using FACS.

Five weeks post-transfer all animals showed engraftment of ILC2 in meninges (FIG. 2), lung, and femur bone marrow with engrafted cells adopting differential marker signatures corresponding to adoptive tissue. Microglia from mice with meningeal engraftment of ILC2s displayed reductions in activation markers relative to PBS controls (See, e.g., FIG. 6A-6I). Density of microglia was decreased in hippocampus of ILC2-transferred mice possibly as a result of reduced activation.

### Example 5. IL-10 is produced by ILC2 following alarmin activation

ILC2s in peripheral tissues have been shown to be activated by alarmins (Vannella et al., 2016, Sci Transl Med., 8(337):337ra65). IL-33 was previously shown to activate meningeal ILC2s (Gadani et al., J Exp Med. 2017;214(2):285-296) and was confirmed by absolute counts, Ki-67 labeling, cytokine labeling (data not shown) and whole-mount labeling of dense clusters of Gata3⁺ ILCs in sinuses of IL-33-treated mice (FIG. 1E).

To interrogate the full transcriptome of mILC2, mice (n=20/group and 2 independent groups) were injected with PBS, rIL-25 or rIL-33 (both, 0.033 mg/kg). mILC2 were then sorted, RNA extracted, and RNAseq analysis performed. Hierarchical clustering showed clear differences in treatment vs. PBS groups. As expected from purity tests, hierarchical clustering reflected prevalence of canonical ILC2-associated factors (FIG. 1F, 1G-m). However, surprisingly, IL-10 emerged above even these, with >11 log2-fold upregulation (FIG. 1Gᵢ₋ᵢᵢᵢ). IL-10 production by meningeal ILC2s was unanticipated. It was reported of IL-10 production by tissue-restricted non-canonical novel innate subsets termed “ILC_{REG}” in gut and “ILC2₁₀” in lung (Seehus et al., 2017; Wang et al., 2017). However, in contrast to the Id3 dependence described for ILC_{REG}, results described herein indicated Id2 dominance and lack of Id3 (FIG. 1G1); similarly, robust Il13 transcript levels (FIG. 1F, 1Gᵢᵢ) diverged from lung-restricted ILC2₁₀, which reportedly lack Il13 expression. In general, meningeal ILC2s mapped unambiguously to canonical ILC2 identity—rather than a novel lineage—by both transcriptome and phenotypic markers.

In order to confirm transcriptional data, IL-10 protein production was probed using several methodologies. First, meningeal ILC2s were sorted from IL-33 treated mice, maintained in culture with IL-2 and IL-7, and then re-stimulated with IL-33. FACS analysis showed these cells to be ST2-expressing and strongly positive for IL-5, IL-13, and also IL-10 (FIG. 1H); Luminex analysis of culture supernates confirmed IL-10 release (FIG. 1I). To rule out the possibility of cell culture artefacts, cells were next isolated acutely from IL-33 treated mice, revealing a significant portion of meningeal ILC2s as IL-10 positive by intracellular labeling (FIG. 1J). Finally, two-photon time-lapse microscopy was performed on acute ex vivo preparations of meninges from IL-33-treated Rag2^{-/-} mice using IL-10 antibody capture reagents. CD90⁺cells showed accumulation of anti-IL-10 antibody fluorescence over time in living tissue, (FIG. 1K) consistent with direct IL-10 capture following release.

In the above experiments, ILC2s were also acutely isolated from non-meningeal tissues for comparison. Surprisingly, ILC2s from calvarium, tibia, and lung also showed positive labeling for IL-10 (data not shown) suggesting that IL-10 production by ILCs might not be tissue-restricted as previously suggested. Based on these observations, human ILC2s were tested for IL-10 competence. To do so, CD45⁺Lin⁻CD11c⁻FcϵR1⁻CD127⁺CRTH2⁺CD161⁺ cells were sorted from healthy donor blood and cultured in conditions analogous to murine ILC2s. Subsequent analysis of supernates showed IL-10 release following IL-33 stimulation (FIG. 1K&1L), showing human ILC2s as indeed capable of IL-10 production. Overall, these results offer several lines of evidence that meningeal ILC2s produce IL-10 following IL-33 stimulation; furthermore, these data suggest that IL-10 production might be a heretofore unrecognized property common to most ILC2s.

### Example 6. ILC2-Derived Factors Suppress Microglial LPS Response

Next, mILC2 and microglia were co-cultured together to determine whether IL-10 production by ILC2 suppresses microglial activation. First, ILC2 were sorted from IL-33-treated mice and microglia were sorted from naive mice. Then, microglia were cultured either alone, or with ILC2, or with ILC2 supernatant, followed by LPS stimulation. Soluble ST2 was included in all conditions at a concentration of 300 ng/ml in order to bind and block direct effects of IL-33 still present in supernates on microglia, which also express the receptor for IL-33.

IL-5, IL-13 and IL-10 protein levels, as measured by Luminex, were higher in wells that contained either ILC2 or ILC2 supernates, compared to wells containing only microglia with/without LPS. Levels of both IL-10 and IL-13 were reduced in the microglia treated with supernates and LPS compared to supernatant alone, suggesting active consumption and receptor binding of IL-10 and IL-13 by activated microglia. This interpretation was reinforced by the observation that no differences were seen in IL-5 levels, for which microglia express no receptor. Several other cytokines and chemokines were also analyzed, with some showing near-complete suppression (e.g. IL-6) by ILC2 or ILC2 supernates and other partial (TNF-a) or no (CXCL10) suppression, suggesting specificity rather than simply a factor toxic to microglia. To specifically determine IL-10-mediated effects, experiments were repeated with both IL-10 (300 ng/ml) and IL10ra (300 ng/ml) neutralizing antibodies added for 24 hours. IL-10 neutralization abolished suppression of microglial response as evidenced by a re-emergence of elevated cytokine and chemokine levels. This result strongly suggested ILC2-derived IL-10 was primarily responsible for the observed microglial suppression.

### Example 7. Human ILC2 Cells Produce IL-10

To confirm that human ILC2 cells could produce IL-10 as seen in murine ILC2, similar experiments with human cells. ILC2 (Lin-IL7ra+CRTH2+CD161+) were sorted from six unique normal human blood donors and cultured in conditions analogous to murine ILC2, with and without IL-33 stimulation, and supernatants were analyzed for cytokine content using Luminex bead assay. ILC2 from five of six donors displayed increased levels for IL-10 (and IL-13, as a positive control) following IL-33 stimulation. This suggested that human ILC2s were capable of IL-10 production. To probe whether ILC2 were unique in the ability to produce IL-10, further samples were obtained but this time sorted for ILC1, 2, and 3. Cells were incubated in media containing factors as appropriate to reinforce ILC1, 2, or 3 subtype (IL-12+IL-15, IL-33, and IL-1b+IL-6+IL-23, respectively). Cytokine levels in the supernatants of all three cell types were measured as before. Wells containing ILC2 showed significantly more IL-10 and IL-13 than wells containing either ILC1 or 3. ILC3 supernatants from one donor showed IL-10, suggesting the possibility that ILC3 might also be IL-10 producers. These results provided additional evidence that human ILCs, and particularly ILC2, were capable of IL-10 production.

Example 8. ILC2 Secreted Factors Suppress Microglial Inflammation; IL-10 Neutralization Abolishes Suppression

ILC2s from IL-33-treated mice and microglia from naive mice were isolated test the ability of ILC2s to suppress microglial inflammatory factors. Microglia was cultured either alone, with ILC2s directly, or with supernates from ILC2s, followed by medium containing R848, a TLR7/8 agonist (similar to IMQ and well-suited to cell culture), or agonist-free control medium. Soluble ST2 was included in order to block any possible direct effects on ST2+microglia by IL-33 from ILC2 supernates. IL-5, IL-13 and IL-10 were all measured from wells containing either ILC2s (Extended data) or ILC2 supernates but were undetectable (IL-5, IL-13) or barely detectable (IL-10) in supernates from wells containing only microglia (FIG. 5A), showing microglia were not a significant source of these factors. Several other cytokines and chemokines were also measured, with a heterogeneous pattern of suppression suggesting specificity (FIG. 5B). As both ILC2s and supernates displayed similar effects, it was concluded that soluble factors likely superseded direct cell contact. In addition to pro-inflammatory cytokines, microglia produce matrix metalloproteinases (Mmps) and in particular, Mmp9, a key player in neurotoxicity and BBB25-27 via enzymatic degradation of extracellular matrix components. In turn, Mmps are counter-regulated by members of the tissue inhibitor of metalloproteinase (Timp) family. Timp 1 message was highly (6.7 Log2Fold, adj p<0.02) upregulated in activated ILC2s according to RNAseq and confirmed by protein analysis of supernates from IL-33 activated ILC2s (FIG. 5C). As expected, microglia showed strongly increased production of Mmp9 following R848 challenge (FIG. 5D) which was then potently suppressed by supernates from activated ILC2s (FIG. 5E).

To probe the importance of IL-10 in suppression of these factors by ILC2 supernates, neutralizing antibodies to IL-10 and IL-10rα were next included in the assays. Near-complete ablation of IL-10 signal in antibody-only-treated wells indicated antibody blockade was effective (FIG. 5F). As such, IL-10 neutralization abolished suppression as evidenced by re-emergence of elevated cytokine and chemokine (FIG. 5G) levels. The relationship between ILC2s, microglia, and regulation of Mmp9 was, as expected, somewhat more complex in terms of IL-10 and Timp1. It was shown in several systems that production of Mmp9 is negatively regulated by IL-10 directly, but also by Timp1, which itself can be induced by IL-1031. Timp1 was also shown to act in an autocrine as well as paracrine fashion. Therefore, the combination of IL-10 and Timp1 would be expected to show superior Mmp9 suppression to IL-10 in the absence of Timp1. In support of this, rmIL-10 indeed suppressed Mmp9, but to a lesser degree than complete ILC2 supernatant (FIG. 5H). In a further test of this possibility, the effects of supernates from ILC2s from Il10 −/− mice with ILC2s from wild type mice on unstimulated and R848-stimulated microglia were compared in terms of total Timp1 protein secretion and it was found that the presence of IL-10 led to increased Timp1 levels in either condition (FIG. 5I). Synergy between IL-10 and Timp1 is suggested by the Mmp9/Timp1 ratio, wherein IL-10 and IL-10ra blockade are sufficient to strongly abrogate suppression mediated by ILC2 supernates (FIG. 5J).

Overall, these data suggest that meningeal ILC2s likely function in multiple ways to suppress not only pro-inflammatory cytokines and chemokine production but also degradation of the BBB by Mmps, including Mmp9. IL-10 is further suggested to play a dual role as a suppressor of Mmp9 directly and also indirectly through Timp1. Other factors highlighted by RNAseq data as upregulated in activated meningeal ILC2s, e.g. amphiregulin, arginase, can also play a role in BBB protection and suppression of neuroinflammation and bear further investigation.

### Passively-Transferred ILC2s Engraft in Meninges and Show IL-10 Dependent Suppression of Neuroinflammation

To test whether ILC2s alone could influence microglial phenotype and suppress neuroinflammation in vivo, and to rule out the possibility that differences observed between Rag2^{-/-} and Rag2^{-/-}γc^{-/-} mice were due either to developmental differences or to microglial deficiency of the IL-2 receptor common γchain, i.v. adoptive transfer of ILC2s into adult Rag2^{-/-} and Rag2^{-/-}γc^{-/-} mice was performed. Robust meningeal engraftment of CD45+Lin−CD9O+ST2+GATA3+cells was confirmed (FIG. 6A) and functionality of engrafted meningeal ILC2s was established by positive detection of IL-5 in supernates of cultured meninges from ILC2-transferred Rag2^{-/-}γc^{-/-} mice but not controls (FIG. 6B). In subsequent examination of acutely isolated brains, side scatter was significantly decreased (FIG. 6C-D) and expression of microglial activation markers was markedly attenuated (FIG. 6D) compared to PBS-treated ILC-deficient controls. These data suggested ILC2s alone were sufficient to modulate microglial phenotype at baseline, thus we wanted to test whether engrafted ILC2s could suppress neuroinflammation, particularly given the ramifications for possible ILC2-based therapeutic applications.

To examine this, adoptive transfer of ILC2s prior to IMQ challenge was also conducted. When whole brain samples were sorted for myeloid cells, microglial (CD11b+CD45Mid) numbers showed no differences between groups (data not shown), whereas brains from ILC2-engrafted mice contained significantly fewer monocytes (CD11b+CD45HiLy6cHi) than brains from ILC-deficient controls, suggesting that ILC2 replenishment was sufficient to blunt infiltration (FIG. 6E). Equivalent numbers per mouse of microglia were also sorted from brains, placed in culture, and supernates analyzed for secreted factors revealing that microglia from brains of ILC2-engrafted mice displayed a suppressed profile—primarily in terms of chemokine levels (FIG. 6F).

Moreover, to determine whether blockade of IL-10 would affect the ability of ILC2 transfer to inhibit cell infiltration and microglial inflammation the above experiments were repeated but with injection of neutralizing antibodies to IL-10 on days 2 and 3 of IMQ treatment. Similar to in vitro experiments, IL-10 blockade abolished the suppressive influence of ILC2 transfer on microglial release of pro-inflammatory factors (FIG. 8). Finally, mice were adoptively transferred with either wild type or Il-10^{-/- ILC}2s to interrogate the importance of ILC2-derived IL-10 in a cell-specific fashion. Again, as suggested by in vitro results, wild type—but not Il10^{-/- ILC}2 transfer blocked both Evans Blue dye (FIG. 6G) and parenchymal infiltration by monocytes (FIG. 6H). In line with these observations, microglial supernates showed suppression of immune factors from mice receiving wild type ILC2s, whereas microglia isolated from mice transferred with Il10^{-/- ILC}2s showed little evidence of suppression (FIG. 6I) suggesting the importance of ILC2-derived IL-10.

In sum, these results suggested that ILC2-secreted factors can play one of perhaps several mechanistic roles in fortification of the barriers protecting delicate CNS tissues, and in suppression of associated microglial inflammation. Indeed, such activity would be consistent with ILC-mediated barrier regulation shown in peripheral mucosal tissues like gut, in which IL-10 also plays a prominent role.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

I/we claim:

1. A method for reducing microglial activation in a subject in need thereof, comprising administering to the subject a therapeutically effective amount of activated type II ILC2s.

2. The method of claim 1, wherein the activated ILC2s are activated by contacting the ILC2s with at least one cytokine selected from the group consisting of IL-33, IL-25, IL-2 and IL-7, or a combination thereof

3. The method of claim 1, wherein the ILC2s are genetically modified, preferably the ILC2s are genetically modified for increased IL-10 production compared to otherwise identical unmodified cells.

4. The method of claim 1, wherein the therapeutically effective amount of activated ILC2s is administered to the subject intravenously or intrathecally.

5. The method of claim 1, wherein the ILC2s are meningeal ILC2s.

6. A method for reducing microglial activation in a subject in need thereof, comprising administering to the subject a therapeutically effective amount of an agent capable of increasing the number of activated type II ILC2s in the subject.

7. The method of claim 6, wherein the agent increases the number of meningeal ILC2s in the subject.

8. The method of claim 6, wherein the agent is IL-33, IL-25, thymic stromal lymphopoietin, or a combination thereof.

9. A method for reducing microglial activation in a subject in need thereof, comprising administering to the subject an effective amount of an agent capable of increasing production of IL-10 by an activated type II ILC2 in the subject.

10. The method of claim 9, wherein the agent increases production of IL-10 by an activated meningeal ILC2 in the subject.

11. The method of claim 9, wherein the agent is IL-33, IL-25, IL-2, IL-4 or a combination thereof.

12. A method for treating a disorder related to microglial activation or increased permeability of blood-brain barrier, comprising administering to a subject in need thereof a therapeutical) effective amount of activated type II ILC2s.

13. The method of claim 12, wherein the subject is in need of a treatment of a neurodegenerative disease, inflammatory disorder, neuropsychologic disorder, chronic pain, traumatic brain injury, spinal cord injury, optic nerve inflammation, or a viral or bacterial infection.

14. The method of claim 13, wherein the subject is in need of a treatment of a neurodegenerative disease selected from the group consisting of Alzheimer' s disease, Parkinson's disease, dementia, multiple sclerosis, a prion disease, amyotrophic lateral sclerosis, Huntington's disease, aging, meningitis and stroke.

15. The method of claim 13, wherein the subject is in need of a treatment of a neuropsychologic disorder selected from the group consisting of depression, anxiety, bipolar depression, and schizophrenia.

16. A method for reducing microglial activation in a subject in need thereof, comprising administering to the subject a therapeutically effective amount of an agent capable of increasing production of Timp 1 by an activated type II ILC2 in the subject.

17. A method for reducing blood-brain barrier permeability in a subject in need thereof, comprising administering to the subject a therapeutically effective amount of activated type II ILC2s, an agent capable of increasing the number of activated type II ILC2s in the subject, or an agent capable of increasing production of IL-10 or Timp1 by an activated type II ILC2 in the subject.

18. A pharmaceutical composition for reducing microglial activation in a subject in need thereof, comprising a therapeutically effective amount of an isolated activated type II ILC2s or genetically modified activated ILC2s and a pharmaceutically acceptable carrier.

19. A pharmaceutical composition for reducing microglial activation in a subject in need thereof, comprising a therapeutically effective amount of an agent capable of increasing the number of activated type II ILC2s in the subject and a pharmaceutically acceptable carrier.

20. A pharmaceutical composition for reducing microglial activation in a subject in need thereof, comprising a therapeutically effective amount of an agent capable of increasing production of IL-10 by an activated type II ILC2 in the subject and a pharmaceutically acceptable carrier.

21. A pharmaceutical composition for reducing microglial activation in a subject in need thereof, comprising a therapeutically effective amount of an agent capable of increasing production of Timp1 by an activated type II ILC2 in the subject and a pharmaceutically acceptable carrier.
